(19) United States
(12) Patent Application Publication
(10) Pub. No.: US 2026/0337622 A1
(43) Pub. Date: Oct. 29, 2026

(54) METHOD FOR PRODUCING BIOLUMINESCENT PLANTS

(71) Applicant: WOODLIGHT, Illkirch-Graffenstaden (FR)

(72) Inventor: Rose-Marie VESIN-AUCLAIR, Illkirch-Graffenstaden (FR)

(73) Assignee: WOODLIGHT, Illkirch-Graffenstaden (FR)

(21) Appl. No.: 19/687,615

(22) Filed: May 26, 2026

Related U.S. Application Data
(63) Continuation-in-part of application No. 17/791,253, filed on Jan. 6, 2021, now Pat. No. 12,662,682.

(30) Foreign Application Priority Data
Jan. 7, 2020 (FR) FR2000104

(51) Int. Cl. C12N 15/82; C12N 9/02; C12N 9/10; C12N 9/12; C12N 9/14

(52) U.S. Cl. CPC C12N 15/8207; C12N 9/0004; C12N 9/1029; C12N 9/1288; C12N 9/14; C12N 15/8206; C12N 15/8213; C12Y 207/08007

(57) ABSTRACT

The present invention relates to a method of making bioluminescent plants by introducing luciferase genes and luciferin biosynthesis genes into plant chloroplasts.

### CROSS-REFERENCE TO RELATED APPLICATIONS

This application is a continuation-in part of U.S. application Ser. No. 17/791,253, filed on Jul. 7, 2022, which is a national stage application under 35 U.S.C. 371 of PCT/EP2021/050103, filed Jan. 6, 2021, which claims benefit of French Application No. FR2000104, filed Jan. 7, 2020, the entire contents of each of which are incorporated herein by reference.

### REFERENCE TO A SEQUENCE LISTING

In accordance with 37 CFR § 1.831, the present specification makes reference to a Sequence Listing submitted electronically as a .xml file named “SequenceListing.xml”. The .xml file was generated on “May 19, 2026” and is “76,633” bytes in size. The entire contents of the electronic Sequence Listing are hereby incorporated by reference.

The field of the invention is plant biology.

Bioluminescence is the production and emission of light by a living organism via a chemical reaction in which chemical energy is converted into light energy.

The basic substrate for luminescence is luciferin, which emits light by oxidation through the action of the enzyme luciferase. In bacteria, the expression of genes related to bioluminescence is controlled by the lux operon. Other organisms are also capable of bioluminescence, such as certain fungi or multicellular beings, including a large number of marine species.

There is a large number of natural luciferases found in species from very different environments. They are found in different families of insects such as the firefly (Photinus pyralis) or in a family of beetles, the click beetle, or in worms (Phrixothrix hirtus). Luciferases are also found in different marine species (Renilla reniformis, Gaussia princeps, Cypridina hilgendorfii . . . ) and in some bacteria (Photobacterium phosphoreum or Vibrio harveyi). This kind of mechanism is also found in some fungi (e.g. Panellus stipticus) or in phytoplankton (Haddock et al., Ann Rev Mar Sci. 2010; 2:443-93; Wilson and Hastings, Annu Rev Cell Dev Biol. 1998; 14:197-230).

The chemical structure of luciferin varies from species to species and can be referred to as coelenterazine. Identification of the metabolic pathway for coelenterazine or luciferin synthesis is important to enable the development of bioluminescent systems to which luciferin need not be added to achieve the bioluminescent effect.

Kotlobay et al (Proc Natl Acad Sci USA. 2018 Dec. 11; 115 (50): 12728-12732) described mechanisms of luciferin formation and recycling in fungi and identified three enzymes in addition to luciferase (Luz). Hispidin synthase (HispS) converts caffeic acid to hispidin, which in turn is a substrate for the enzyme hispidin-3-hydroxylase (H3H), which converts hispidin to luciferin (3-hydroxyhispidin). Finally, caffeylpyruvate hydrolase (CPH) converts oxyluciferin (caffeil-pyruvic acid), obtained after oxidation of luciferin by luciferase, to caffeic acid. However, the authors did not describe that this system can be used in plants in general and chloroplasts in particular.

Mitioushkina et al (BioRxiv, 2019, doi.org/10.1101/809376) described that expression of the Neonothopanus nambi HispS, H3H, and Luz genes in tobacco cells (integration into the nuclear genome) generated luminescent plants. The authors did not use the NpgA gene.

The teaching of WO2020005120A1 is similar to that of the above two documents.

Krichevsky et al (PLOS ONE 5 (11): e15461) describe self-luminescent plants using the lux operon of Photobacterium leiognathi (luxCDABEG) to transform chloroplasts. The different transgenes used (spectinomycin and streptomycin antibiotic resistance gene (aadA) and lux operon (luxCDABEG)) are under the control of the same chloroplast promoter. Thus, 7 genes were introduced as an operon under the control of a single chloroplast promoter. WO2009017821 shows the same teaching as the previous document.

Bioluminescent plants can be used in the field of decoration and events. Preferably, such plants should be able to produce light autonomously, or after providing a low cost substrate. The amount of light that needs to be produced should be sufficient for the effect to be aesthetically pleasing. It is also preferred that these plants are sterile to avoid any contamination in the environment.

In order to address the above issues, the Applicant has developed a plant in which at least one chloroplast of a cell contains the genes encoding the proteins hispidin-3-hydroxylase (H3H, GenBank: BBH43497.1) and luciferase (Luz, GenBank: BBH43509.1) under the control of promoters active in the chloroplast. Thus, the chloroplast contains an expression cassette for the H3H and Luz proteins in chloroplasts.

In one particular embodiment, a plurality of chloroplasts of the cell contains the expression cassette. In another embodiment, a plurality of cells of the plant contains at least one chloroplast containing the expression cassette. In a particular embodiment, all cells of the plant contain at least one chloroplast containing the expression cassette. In this embodiment, at least 50% of the chloroplasts of the plant contain the expression cassette, i.e., if one takes a portion of the plant and investigates the presence of the expression cassette in the chloroplasts of that portion of the plant, at least 50% contain that cassette. This can be easily verified by any method known in the art such as quantitative PCR, the preferred method.

It is interesting to process plant chloroplasts (or plastids) for several reasons:
these are present in large quantities in each cell, which allows to multiply the quantity of enzymatic reaction points producing bioluminescence
the chloroplasts are present in the cytoplasm of the cell and are not transmissible by pollen, which avoids contamination to other plants of the same species
there are several copies of the genome in each chloroplast, which also allows to multiply the amount of enzymatic reaction points producing bioluminescence.

### Transformation of Chloroplasts

Although methods for plastid transformation are known in the art, chloroplast transformation is, in general, much more complicated, time consuming and with a much lower success rate than nuclear transformations. This is especially true when large or multiple genes are to be integrated.

Chloroplasts can be transformed by biolistics. The meristematic tissues (cells) are bombarded with gold microbeads coated with DNA. After divisions, the number of transformed plastids will increase more rapidly than the non-transformed plastids (especially when using a selection medium), and the non-transformed plastids will be “lost” by dilution.

Alternatively, the PEG (polyethylene glycols) method can be used. The destabilization of plasma membranes in the presence of PEGs allows the entry of transgenes into chloroplasts.

### Transgenes

The invention thus relates to a plant in which at least one chloroplast of a cell contains genes encoding the proteins hispidin-3-hydroxylase (H3H) and luciferase (Luz) under the control of promoters active in the chloroplast.

The invention also relates to a plant cell in which at least one chloroplast of a cell contains genes encoding the proteins hispidin-3-hydroxylase (H3H) and luciferase (Luz) under the control of promoters active in the chloroplast. It is possible to regenerate a complete plant from this cell.

The cell so described can produce light by adding hispidin to the culture medium. When this cell is present in a plant, the hispidin present in the culture medium reaches the cell via the sap.

In the chloroplast, hispidin is then converted to luciferin by the enzyme H3H, and then luciferin is oxidized to oxyluciferin by luciferase Luz, producing light. This method allows luminescence to be obtained only when the substrate (hispidin) is supplied to the plant.

In another embodiment, the chloroplasts of plant cells contain, in addition to the genes encoding the H3H and Luz enzymes, a gene encoding the hispidin synthase enzyme HispS, under the control of a promoter active in the chloroplasts. In this embodiment, luminescence will be initiated by caffeic acid naturally present in the plant cell (cytoplasm) and in the chloroplast. It is then transformed into hispidin, which then gives luciferin. Alternatively, luminescence can be initiated by adding caffeic acid to the culture medium.

In this embodiment, the cell can thus produce luminescence in an autonomous way, i.e. it contains all the genes allowing the synthesis of luciferin, as well as its recycling.

In another embodiment, however, it is preferred that it contains all the genes for the synthesis of luciferin, as well as the genes for the recycling of caffeoyl-pyruvic acid, the reaction product of luciferase on luciferin, in order to avoid its accumulation and a risk of caffeic acid deficiency.

In this embodiment, the chloroplast contains genes encoding the proteins
caffeoyl pyruvate hydrolase (CPH, GenBank: BBH43519.1), hispidin synthase (HispS, GenBank: BBH43485.1), under the control of promoters active in the chloroplasts.

In a particular embodiment, the luciferase sequence is SEQ ID NO: 20 (with or without the poly_histidine tag, His-tag).

In a particular embodiment, the sequence of the H3H enzyme is SEQ ID NO: 21 (with or without the poly_histidine tag, His-tag).

In a particular embodiment, the sequence of the CPH enzyme is SEQ ID NO: 22 (with or without the poly_histidine tag, His-tag).

In a particular embodiment, the sequence of the HispS enzyme is SEQ ID NO: 23 (with or without the poly_histidine tag, His-tag).

In one embodiment, the chloroplast also contains a gene encoding a phosphopantetheinyl transferase (NpgA, NCBI Reference Sequence: XP_663744.1), also under the control of a promoter active in plastids. The gene encoding NpgA can be added for each embodiment as described above.

In a particular embodiment, the sequence of NpgA is SEQ ID NO: 24 (with or without the poly_histidine tag, His-tag).

In a particular embodiment, the plant is such that all of its cells contain at least one chloroplast (and preferably at least 50% of its chloroplasts) transformed by the genes encoding H3H, Luz, CPH, HispS and NpgA proteins.

The invention also relates to a plant cell in which at least 50% of the chloroplasts are transformed by the genes encoding the H3H, Luz, CPH, HispS and NpgA proteins.

### Vectors

The invention also relates to a vector containing
(a) an origin of replication in a bacterium or yeast, preferably an origin of replication in Escherichia coli,
(b) a nucleic acid sequence encoding the Luz protein (in particular SEQ ID NO: 1 or 28) under the control of a promoter functional in chloroplasts,
(c) a nucleic acid sequence encoding the H3H protein (in particular SEQ ID NO: 2 or 32) under the control of a promoter functional in chloroplasts,
(d) two nucleic acid sequences present in a chloroplast, preferably trnI (SEQ ID NO: 6 or 25 or 35) and trnA (SEQ ID NO: 7 or 26 or 36), flanking said nucleic acid sequences (b) and (c),
these sequences (b) and (c) being thus located between these two sequences (d).

In a particular embodiment, the vector also contains
(e) a nucleic acid sequence coding for a Cph protein (in particular SEQ ID NO: 3), located between sequences (d) with sequences (b) and (c).

In a particular embodiment, the vector also contains
(f) a nucleic acid sequence encoding a HispS protein (in particular SEQ ID NO: 4), located between sequences (d) with sequences (b) and (c).

In a particular embodiment, the vector also contains
(g) a nucleic acid sequence encoding an NpgA protein (in particular SEQ ID NO: 5), located between sequences (d) with sequences (b) and (c).

In a preferred embodiment, the vector contains the origin of replication (a), as well as the sequences (b), (c), (e), (f) and (g) flanked by the sequences (d).

### Host Cells

The invention also relates to a host cell containing a vector as described above. This cell is preferably a bacterial cell, preferably Escherichia coli transformed by the vector.

### Optimization of Coding Sequences

It is preferable that the nucleic sequence of the genes has been optimized for expression in chloroplasts (adaptation of chloroplast codon usages).

This can be based on the information given in Nakamura et al (Plant J. 2007 January; 49 (1): 128-34. 2006), Liu et al (J Genet. 2005 April; 84 (1): 55-62) or Zhang et al (Journal of Integrative Plant Biology 2007, 49 (2): 246-254). In particular, we use the databases of the Kazusa DNA Research Institute which can be found on their website https://www.kazusa.or.jp/codon.

The sequences SEQ ID NO: 1 to SEQ ID NO: 5, SEQ ID NO: 28 and SEQ ID NO: 32 are thus sequences optimized for expression in chloroplasts. These sequences, or sequences having at least 90% identity, preferably at least 95% identity, more preferably at least 98% identity, most preferably at least 99% identity with these sequences and encoding the proteins SEQ ID NO: 20 to SEQ ID NO: 24 respectively, can be used.

Preferably, the genes are optimized to obtain a GC rate between 35% and 40%. The codons preferentially expressed in the chloroplasts are also chosen, taking into account the data of Liu et al.

### Organization

The system used is a system derived from fungi (fungus). In particular, it is preferred to use the enzyme system from a fungus selected from Neonothopanus nambi, Neonothopanus gardneri, or Omphalotus olearius.

Preferably, it is preferred when all of the enzymes used (HispS, H3H, Luz, and CPH) are from the same organism, preferably Neonothopanus nambi. The use of sequences from Neonothopanus gardneri, which are very similar to those from Neonothopanus nambi, is also considered. Alternatively, some sequences from Neonothopanus nambi and others from Neonothopanus gardneri may be used together.

It is recalled that the npgA (4′-phosphopantetheinyl transferase) gene originates from Aspergillus nidulans.

### Promoters and Coding Sequences

### Operon

In a first embodiment, the different genes are expressed under the control of a single promoter, in the form of an operon, as is known in the art, for genes involved in the same metabolic pathway (cf Saxena et al, 2014 J Biosci. 2014 March; 39 (1): 33-41; Kumar et al, 2012 Metab Eng. 2012 January; 14 (1): 19-28).

In this embodiment, a promoter selected from the Patpl, Prrn, PrbcL, PpsbA, NtPpsbA or PePpsbA promoters (in particular those described by the sequences SEQ ID NO: 8 to SEQ ID NO: 12, SEQ ID NO: 29, SEQ ID NO: 33 and SEQ ID NO: 37) can be used.

However, this embodiment is not preferred.

### Individual Sponsors

In another embodiment, each transgene introduced into the chloroplast of the plant is under the control of its own promoter (a promoter of its own, it being understood that the same promoter can be used for two genes, but that it is preferred when at least two transgenes are under the control of two different promoters). In this embodiment, the transgenes are therefore not expressed within an operon. This is a preferred embodiment. Indeed, the fact of assigning to each gene an optimal regulation system (promoters and terminators) that is specific to it, allows to increase the expression levels, as compared to the use of a system based on an operon. This mode of operation thus goes against the modes generally observed in the art, summarized in Boehm and Bock (Plant Physiol. 2019 March; 179 (3): 794-802), which refers to recent research for optimal expression of several proteins in chloroplasts.

This paper shows that as soon as simultaneous expression of two or more proteins is desired, genes are arranged as an operon such as for the expression of: polyhydroxybutyrate (Bohmert-Tatarev et al., Plant Physiol. 2011 April; 155 (4): 1690-708), insect proteins (De Cosa et al., Nat Biotechnol. 2001 January; 19 (1): 71-4) 7, or carotenoids (Hasunuma et al., Plant J. 2008 September; 55 (5): 857-68), but also for the expression of entire metabolic pathways such as those of: vitamin E (Lu et al., Proc Natl Acad Sci USA. 2013 Feb. 19; 110 (8): E623-32), artemisinic acid (Saxena et al., J Biosci. 2014 March; 39 (1): 33-41, Fuentes et al, eLife. 2016; 5: e13664), mevalonate (Kumar et al., Metab Eng. 2012 January; 14 (1): 19-28, Saxena et al., op. cit.) and dhurrin (Gnanasekaran et al., J Exp Bot. 2016 April; 67 (8): 2495-506).

Furthermore, Boehm and Bock (op. cit.) also recall the latest advances in improving the efficiency of synthetic operons; by adding intercistronic expression elements (IEEs) (Zhou et al., Plant J. 2007 December; 52 (5): 961-972), or by stabilizing messenger RNAs with proteins that bind to them (PRRs), (Legen et al., Plant J. 2018 April; 94 (1): 8-21). Even more recently, they recall that Fuentes et al. (op. cit.) have shown that the complexity and number of accessible pathways have been extended by a trick that combines chloroplast transformation with nuclear transformation (“combinatorial supertransformation of transplastomic recipient lines (COSTREL)”). These authors classically transformed tobacco plant chloroplasts with the artemisinic acid metabolic pathway (as an operon) and the nucleus of these plants once transformed with five genes (CYB5, ADH1, ALDH1, DBR2, DXR). With this approach, the authors improved the production of artemisinic acid by 77 times.

Thus, the art proposes rather to use operons and to optimize them (manufacture of synthetic operons), when one wishes to express several genes in chloroplasts, in particular when the genes considered must cooperate with each other in a given metabolic pathway.

In particular, the promoters Patpl, Prrn, PrbcL, PpsbA, NtPsbA or PePpsbA can be chosen.

Preferably, the H3 h gene is under the control of the PpsbA promoter, in particular the portion specified in SEQ ID NO: 9 (synthetic construct). Preferably, the H3 h gene is under the control of the NtPpsbA promoter, in particular the portion specified in SEQ ID NO: 33 (native promoter from Nicotiana). Preferably, the H3h gene is under the control of the PePpsbA promoter, in particular the portion specified in SEQ ID NO: 37 (native promoter from Petunia).

Preferably, the Luz gene is under the control of the PpsbA promoter, in particular the portion specified in SEQ ID NO: 9. Preferably, the Luz gene is under the control of the Prrn promoter, in particular the portion specified in SEQ ID NO: 29.

Preferably, the Cph gene is under the control of the PrbcL promoter, in particular the portion specified in SEQ ID NO: 10.

Preferably, the HispS gene is under the control of the Prrn promoter, in particular the portion specified in SEQ ID NO: 11.

Preferably the npgA gene is under the control of the Patpl promoter, in particular the portion specified in SEQ ID NO: 12.

In one particular embodiment, sequences, and in particular promoters, are optimized by selecting particular 5′UTR sequences to optimize gene expression in chloroplasts (De Costa et al. Genes Genet Syst. 2001 December; 76 (6): 363-71); Drechsel and Bock Nucleic Acids Res. 2011 March; 39 (4): 1427-38); Shinozaki and Sugiura Gene. 1982 Nov. 20 (1) 91-102 and Nucleic Acids Res. 1982 Aug. 25; 10 (16): 4923-34; Kuroda and Maliga, Plant Physiol. 2001 January; 125 (1): 430-6).

Sequences SEQ ID NO: 9 to SEQ ID NO: 12, SEQ ID NO: 29, SEQ ID NO: 33 and SEQ ID NO: 37 represent such optimized promoters with added 5′UTRs to enhance expression.

Thus, preferably, the H3 h gene is under the control of the optimized PpsbA promoter described by SEQ ID NO: 9. Preferably, the H3 h gene is under the control of the optimized NtPpsbA promoter described by SEQ ID NO: 33. Preferably, the H3 h gene is under the control of the optimized PePpsbA promoter described by SEQ ID NO: 37.

Preferably, the Luz gene is under the control of the optimized PpsbA promoter described by SEQ ID NO: 9. Preferably, the Luz gene is under the control of the optimized Prrn promoter described by SEQ ID NO: 29 (which comprises 17 nucleic acids of the 5′UTR sequence derived from the rbcL gene).

Preferably, the Cph gene is under the control of the optimized PrbcL promoter described in SEQ ID NO: 10.

Preferably, the HispS gene is under the control of the optimized Prrn promoter described in SEQ ID NO: 11 (which comprises 58 nucleic acids of the 5′UTR sequence derived from the rbcL gene).

Preferably the npgA gene is under the control of the optimized Patpl promoter described by SEQ ID NO: 12.

Terminators positioned 3′ to the coding nucleic sequences are also used. The terminators represented by the sequences SEQ ID NO: 13 to SEQ ID NO: 18, SEQ ID NO: 31 and SEQ ID NO: 34 can be used.

In a preferred embodiment, a (5′-3′) SEQ ID NO: 9-SEQ ID NO: 1-SEQ ID NO: 14 construct is used to express Luz. In a preferred embodiment, a (5′-3′) SEQ ID NO: 29-SEQ ID NO: 28-SEQ ID NO: 31 construct is used to express Luz.

In a preferred embodiment, a (5′-3′) SEQ ID NO: 9-SEQ ID NO: 2-SEQ ID NO: 15 construct is used to express H3H. In a preferred embodiment, a (5′-3′) SEQ ID NO: 33-SEQ ID NO: 32-SEQ ID NO: 34 construct is used to express H3H. In a preferred embodiment, a (5′-3′) SEQ ID NO: 37-SEQ ID NO: 32-SEQ ID NO: 34 construct is used to express H3H.

In a preferred embodiment, a (5′-3′) SEQ ID NO: 10-SEQ ID NO: 3-SEQ ID NO: 16 construct is used to express Cph.

In a preferred embodiment, a (5′-3′) SEQ ID NO: 11-SEQ ID NO: 4-SEQ ID NO: 14 construct is used to express HispS.

In a preferred embodiment, a (5′-3′) SEQ ID NO: 12-SEQ ID NO: 5-SEQ ID NO: 18 construct is used to express NpgA.

In a preferred embodiment, a (5′-3′) SEQ ID NO: 8-SEQ ID NO: 19-SEQ ID NO: 17 construct is used to express the aadA selection gene.

Integration of the Expression Cassette within the Chloroplasts

As seen above, in a particular embodiment, all the genes present in the chloroplasts form an expression cassette, i.e. these genes are present one after the other on a DNA fragment. Thus, the chloroplasts are transformed with this expression cassette, in order to obtain the expression of the genes coded in this expression cassette.

It is preferred when the genes are integrated into the chloroplast genome. In particular, homologous recombination is used to introduce the genes at a selected location in the chloroplast genome.

Many insertion sites are possible in the chloroplast genome. The preferred choice is to integrate the expression cassette in a non-coding region of the chloroplast genome. Once can use inter-gene sequences between two chloroplast transfer RNA coding sequences.

In particular, the trnI (SEQ ID NO: 6 or 25 or 35) and trnA (SEQ ID NO: 7 or 26 or 36) sites, coding for the isoleucine and alanine transfer RNAs, are chosen. One can use sequences SEQ ID NO: 6 or 25 or 35, or SEQ ID NO: 7 or 26 or 36, or sequences containing these sequences. Sequences included in SEQ ID NO: 6 or 25 or 35, or SEQ ID NO: 7 or 26 or 36, can also be used, but in this case, it is preferable to use sequences with at least 1000 bases, preferably at least 1300 bases, preferably at least 1500 bases, preferably at least 1700 bases of SEQ ID NO: 6 or 25 or 35, or SEQ ID NO: 7 or 26 or 36. In fact, in order to increase the chances of homologous recombination, it is preferable to use sequences that are as long as possible.

The sequences SEQ ID NO: 6 and SEQ ID NO: 7 are from tobacco (Nicotiana benthamiana). They are therefore particularly suitable for integration by homologous recombination in tobacco chloroplasts. However, they can be used for other plants, because of the high homology between these sequences and the trnI and trnA sequences of chloroplasts of other plants. Thus, sequences having at least 99% identity, more preferably at least 99.45% identity, more preferably at least 99.5% identity, more preferably at least 99.7% identity with SEQ ID NO: 6 or SEQ ID NO: 7 can be used.

The sequences SEQ ID NO: 25 and SEQ ID NO: 26 are from tobacco (Nicotiana tabacum, Nicotiana sylvestris, Nicotiana glauca). They are therefore particularly suitable for integration by homologous recombination in tobacco chloroplasts. However, they can be used for other plants, because of the high homology between these sequences and the trnI and trnA sequences of chloroplasts of other plants. Thus, sequences having at least 99% identity, more preferably at least 99.45% identity, more preferably at least 99.5% identity, more preferably at least 99.7% identity with SEQ ID NO: 25 or SEQ ID NO: 26 can be used.

The sequences SEQ ID NO: 35 and SEQ ID NO: 36 are from Petunia (Petunia×hybrida Blue Sky, Petunia integrifolia, Petunia exserta and Petunia axillaris). They are therefore particularly suitable for integration by homologous recombination in tobacco chloroplasts. However, they can be used for other plants, because of the high homology between these sequences and the trnI and trnA sequences of chloroplasts of other plants. Thus, sequences having at least 99% identity, more preferably at least 99.45% identity, more preferably at least 99.5% identity, more preferably at least 99.7% identity with SEQ ID NO: 35 or SEQ ID NO: 36 can be used.

### Sequence Comparison/Determination of Percentage Identity

In order to evaluate the identity between two nucleic sequences, the Blastn (nucleotide blast) software developed from Altschul et al, (1997), Nucleic Acids Res. 25:3389-3402; Altschul et al, (2005) FEBS J. 272:5101-5109, available on the NCBI website (https://blast.ncbi.nlm.nih.gov/Blast.cgi) is used using the following parameters, given in English:
Max target sequences: 100
Select the maximum number of aligned sequences to display
Short queries: Automatically adjust parameters for short input sequences
Expect threshold: 10
Word size: 28
Max matches in a query range: 0
Scoring Parameters
Match/Mismatch Scores: 1, −2
Gap Costs: Linear
Filters and Masking
Filter: Low complexity regions filter: on
Mask: Mask for lookup table only: on

In order to evaluate the identity between two protein sequences, the Blastp (protein blast) software developed from Altschul et al, (1997), Nucleic Acids Res. 25: 3389-3402; Altschul et al, (2005) FEBS J. 272:5101-5109, available on the NCBI website (https://blast.ncbi.nlm.nih.gov/Blast.cgi) is used using the following parameters, given in English:
Expected threshold: 10
Word size: 3
Max matches in a query range: 0
Matrix: BLOSUM62
Gap Costs: Existence 11, Extension 1.
Compositional adjustments: Conditional compositional score matrix adjustment
No filter for low complexity regions

### Plant

The plant according to the invention is preferably an ornamental plant.

The plant according to the invention is selected from the genus Nicotiana or Petunia. In particular, the plant according to the invention is selected from Nicotiana tabacum, Nicotiana sylvestris, Nicotiana glauca. In particular, the plant according to the invention is selected from Petunia hybrida, Petunia integrifolia, Petunia exserta or Petunia axillaris.

It is preferably chosen from the group consisting of Hedera helix, Petunia axillaris subsp. axillaris, Nicotiana benthamiana, Ficus benjamina, ficus elastica, Ficus microcarpa, Chlorophytum comosum, Monstera deliciosa, Sansevieria socotrana, Pelargonium×hortorum, Spathiphyllum wallisii, Dracaena draco, Dracaena angustifolia, Yucca aloifolia, Beaucarnea recurvata, Syngonium podophyllum, Fittonia verschaffeltii, Aloe vera, Aloe jucunda, Aloe juvenna, Dieffenbachia. Livistona speciosa, Orchidaceae. In particular, the plant is Nicotiana benthamiana. In another embodiment, the plant is Petunia (Petunia axillaris subsp. Axillaris). In another embodiment, the plant is ivy (Hedera helix).

In view of the above, a general aspect of the invention relates to a plant in which all cells comprising at least one chloroplast are such that the at least one chloroplast comprises at least two constructs each comprising a promoter operably linked to a nucleic acid encoding a protein, wherein the proteins are:
a hispidin-3-hydroxylase (H3H);
a luciferase (Luz),
   said plant being selected from the genus Nicotiana or Petunia.

In another embodiment, the invention relates to the plant as described above, wherein said hispidin-3-hydroxylase (H3H) is a Neonothopanus nambi H3H and/or said luciferase (Luz) is a Neonothopanus nambi Luz.

In another embodiment, the invention relates to the plant as described above, said plant being selected from Nicotiana tabacum, Nicotiana sylvestris, Nicotiana glauca, Petunia hybrida, Petunia integrifolia, Petunia exserta or Petunia axillaris.

In another embodiment, the invention relates to the plant as described above, wherein said at least one chloroplast further comprises three constructs each comprising a promoter operably linked to a nucleic acid encoding a protein, wherein the proteins are:
caffeoyl-pyruvate hydrolase (CPH);
hispidin synthase (HispS); and
phosphopantetheinyl transferase (NpgA),
   said plant being autoluminescent.

In another embodiment, the invention relates to the plant as described above, wherein said caffeoyl-pyruvate hydrolase (CPH) is a Neonothopanus nambi CPH and/or said hispidin synthase (HispS) is a Neonothopanus nambi HispS and/or said phosphopantetheinyl transferase (NpgA) is a Aspergillus nidulans NpgA.

In another embodiment, the invention relates to the plant as described above, wherein the nucleic acids comprise codons adapted for expression in the at least one chloroplast.

In another embodiment, the invention relates to the plant as described above, wherein the promoters are selected from SEQ ID NO: 8, SEQ ID NO: 9, SEQ ID NO: 10, SEQ ID NO: 11, SEQ ID NO: 12, SEQ ID NO: 29, SEQ ID NO: 33 and SEQ ID NO: 37.

In another embodiment, the invention relates to the plant as described above, wherein said at least one chloroplast further comprises three constructs each comprising a promoter operably linked to a nucleic acid encoding a protein, wherein the proteins are:
caffeoyl-pyruvate hydrolase (CPH);
hispidin synthase (HispS); and
phosphopantetheinyl transferase (NpgA),
   said promoters being selected from SEQ ID NO: 8, SEQ ID NO: 9, SEQ ID NO: 10, SEQ ID NO: 11, SEQ ID NO: 12, SEQ ID NO: 29, SEQ ID NO: 33 and SEQ ID NO: 37,
   said plant being autoluminescent.

In another embodiment, the invention relates to the plant as described above, wherein the constructs are integrated into the genome of the at least one chloroplast at the trnI and trnA sites, represented by SEQ ID NO: 6 or 25 or 35 and SEQ ID NO: 7 or 26 or 36 respectively.

In another embodiment, the invention relates to the plant as described above, wherein said at least one chloroplast further comprises three constructs each comprising a promoter operably linked to a nucleic acid encoding a protein, wherein the proteins are:
caffeoyl-pyruvate hydrolase (CPH);
hispidin synthase (HispS); and
phosphopantetheinyl transferase (NpgA),
   said constructs being integrated into the genome of the at least one chloroplast at the trnI and trnA sites, represented by SEQ ID NO: 6 or 25 or 35 and SEQ ID NO: 7 or 26 or 36 respectively,
   said plant being autoluminescent.

In another embodiment, the invention relates to the plant as described above, wherein the constructs have been integrated into the genome of the at least one chloroplast by homologous recombination.

### Production Method

The invention also relates to a method of producing a plant as described above, comprising a step of inserting the transgenes as described above into the genome of chloroplasts of plant cells. The method also preferably includes the step of regenerating a plant by callus culture.

In a first embodiment, the method comprises inserting transgenes encoding the H3H and Luz enzymes into the genome of plant cell chloroplasts.

In another embodiment, the method comprises inserting transgenes encoding Cph, H3H and Luz enzymes into the genome of plant cell chloroplasts.

In another embodiment, the method comprises inserting transgenes encoding the enzymes Cph, HispS, H3H and Luz into the genome of chloroplasts of plant cells.

In another embodiment, the method comprises inserting transgenes encoding the enzymes Cph, HispS, H3H and Luz into the genome of plant cell chloroplasts, as well as a transgene encoding NpgA.

In one embodiment, the integration of transgenes is performed by bombarding plant leaves with a plasmid using a particle gun. To do this, the expression cassette (DNA fragment carrying the transgenes that one wishes to integrate into the chloroplast genome) is prepared, and metal microbeads (preferably gold, but can also be tungsten) are coated, which are then projected onto the plant cells.

In another embodiment, the plant cells are contacted with polyethylene glycol (PEG), which destabilizes the plasma membranes and allows the entry of DNA fragments carrying the transgenes to be integrated into the plastid genome.

It is preferred when the integration of transgenes into the chloroplast genome is performed by homologous recombination. Thus, transgenes are flanked by sequences homologous to sequences of the chloroplast chromosome. The integration of the transgenes is thus performed by the organelle machinery by homologous recombination at the site

In both cases, the transformed cells are cultivated under conditions that produce calluses, which are grown and from which a plant is regenerated by methods known in the art.

In a preferred embodiment, the callus culture is performed on a selective medium. A selective medium is a medium containing a selective element (often an antibiotic or herbicide) on which only cells containing a gene for resistance to the selective element can grow, while cells not containing the gene cannot grow or grow at a slower rate.

Selective elements include antibiotics: neomycin/kanamycin and nptll (aminoglycoside 3′-phosphotransferase), betain and badh (betain aldehyde dehydrogenase), hygromycin B and hph (hygromycin B phosphotransferase), spectinomycin/streptomycin and aadA (aminoglycoside 3′-adenyltransferase), chloramphenicol and cat (chloramphenicol acetyltransferase), amikacin and aphA6 (3′-aminoglycoside phosphotransferase) sulfonamides and sull (dihydropteorate synthase DHPS), gentamycin and aacC1 (gentamycin acetyltransferase) or the herbicides bialophos/phosphinotricin/glufosinate and pat (phosphinotricin actyltransferase) glyphosate and gox (glyphosate oxidoreductase) or epsp (5 eonylpyruvyl shikimate-3-phosphate synthase), bromoxynil and bxn (bromosynil nitrilase), sulfonylureas/imidazolines/triazolopyrimidines/pyrimidylbenzoates and als (acetolactate synthase).

In particular, the aadA (aminoglycoside 3′-adenyltransferase) gene that confers resistance to spectinomycin is used, a coding sequence of which is represented by SEQ ID NO: 19 or 27.

The resistance gene is introduced into the expression cassette containing the transgenes of interest, under the control of a promoter active in chloroplasts. Alternatively, a system can be implemented in which the resistance gene can be excised after transformation, e.g. following the teaching of Scutt et al (Biochemistry 84 (2002) 1119-1126) or Lantham et al (Nature Biotechnology, 2000, (18), 1172-76).

In particular, the resistance gene is inserted between one gene of the transgenes of interest and its promoter, i.e. at the 3′ end of the promoter and the 5′ end of the said one gene of the transgenes of interest. For instance, the resistance gene (aadA) is inserted between the luciferase (Luz) transgene and its promoter. Preferably a (5′-3′) SEQ ID NO: 29-SEQ ID NO: 27-SEQ ID NO: 28(-SEQ ID NO: 31) construct is used to express aadA and Luz. To ensure efficient translation of both aadA and Luz, a T7 phage gene 10 leader sequence (T7g10L, SEQ ID NO: 30) is placed between aadA and luz genes. Preferably a (5′-3′) SEQ ID NO: 29-SEQ ID NO: 27-SEQ ID NO: 30-SEQ ID NO: 28(-SEQ ID NO: 31) construct is thus used to express aadA and Luz.

In view of the above, another general aspect of the invention relates to a method of producing the plant as described above, wherein the method comprises:
i. transforming chloroplasts of Nicotiana or Petunia genera plant cell with the at least two constructs and a selection construct comprising a resistance gene selected from the group consisting of: nptII, badh, hph, aadA, cat, aphA6, aacC1, pat, gox, epsp, bxn and als,
   wherein the at least two constructs and the selection construct are inserted into the genome of the chloroplasts by homologous recombination,
ii. producing callus from the transformed cell grown on a selective medium comprising:
   an antibiotic selected from the group consisting of: neomycin, kanamycin, betain, hygromycin B, spectinomycin, streptomycin, chloramphenicol, amikacin and gentamycin; or
   a herbicide selected from the group consisting of: bialophos, phosphinotricin, glufosinate, glyphosate, bromoxynil, sulfonylureas, imidazolines, triazolopyrimidines and pyrimidylbenzoates; and
iii. regenerating a plant from the callus.

In another embodiment, the invention relates to the method as described above, wherein transformation is performed by bombarding plant leaves using a particle gun.

In another embodiment, the invention relates to the method as described above, wherein transformation is performed by destabilizing plasma membranes with polyethylene glycol (PEG).

In another embodiment, the invention relates to the method as described above, wherein the resistance gene is aadA and the selective medium comprises spectinomycin and/or streptomycin.

In another embodiment, the invention relates to the method as described above, wherein the method further comprises in step i. transforming chloroplasts of Nicotiana or Petunia genera plant cell with additional three constructs each comprising a promoter operably linked to a nucleic acid encoding a protein, wherein the proteins are:
caffeoyl-pyruvate hydrolase (CPH);
hispidin synthase (HispS); and
phosphopantetheinyl transferase (NpgA),
   wherein the additional three constructs are inserted into the genome of the chloroplasts by homologous recombination, said plant being autoluminescent.

The invention also relates to a light system comprising a bioluminescence-emitting plant as described above, i.e., at least one cell of which contains at least one chloroplast containing the above-mentioned genes, and which emits bioluminescence by oxidation of luciferin by the enzyme Luz.

Preferably, the system contains a plant in which at least 50% of the chloroplasts contain the above-mentioned enzyme system.

The invention also relates to a method of producing light, comprising the step of adding hispidin to the culture medium of a plant as described above, and at least one chloroplast of which contains at least the genes encoding the H3H and Luz enzymes (preferably integrated into its genome).

The invention also relates to a method of producing light, comprising the step of spraying hispidin to a plant as described above, and at least one chloroplast of which contains at least the genes encoding the H3H and Luz enzymes (preferably integrated into its genome).

In another embodiment, the invention relates to the method of producing light as described above, wherein the method comprises:
adding hispidin to a culture medium on which the plant as described above is growing; or
spraying hispidin to the plant as described above.

The invention also relates to a method of producing light (by a plant), comprising the step of adding caffeic acid to the culture medium of a plant as described above, and at least one chloroplast of which contains at least the genes encoding the HispS, H3H and Luz enzymes (preferably integrated into its genome).

The invention also relates to a method of producing light (by a plant), comprising the step of adding caffeic acid to the culture medium of a plant as described above, and of which at least one chloroplast contains the genes encoding the HispS, H3H, Luz, Cph enzymes (preferably integrated in its genome).

Preferably the chloroplast also contains the gene encoding NpgA, in the above methods.

### LIST OF FIGURES

FIG. 1. Schematics of transgene constructs for luz h3h insertion in chloroplast genomes of Nicotiana (A) and Petunia plants (B).

FIG. 2. Plant material was put in contact with hispidin (0.1 mM). Luminescence of plant material shown in left panels was recorded using Amersham Image 680 (right panels). Wild-type plants (WT) and a non-transformed callus (−) treated in parallel are shown as negative controls. The vector insertion in the plastome of (+) material was confirmed by Southern blot analyses.

A) Nicotiana tabacum WT and transformed (+) plants

B) Nicotiana sylvestris a WT plant, a non-transformed callus (−) and a transformed callus (+)

C) Nicotiana glauca WT and transformed (+) plants

### EXAMPLES

The following examples and figures describe a particular embodiment of the invention.

### Example 1. Preparation of the Plasmid Containing the H3H and Luz Genes and the Sequences Allowing the Integration of these Genes into the Chloroplast Genome and their Expression

The H3H and Luz gene sequences were adapted with chloroplast usage codons and then synthesized (SEQ ID NO: 2 and SEQ ID NO: 1). The promoters were selected from the set of promoters present in the chloroplast genome and modified in their 5′UTR sequence so as to maximize the expression of genes under their controls. These modified promoters were then synthesized. The terminators were selected from the set of terminators present in the chloroplast genome and some were optimized to be as short as possible while keeping their functions. They were also synthesized. The trnI and trnA sequences (SEQ ID NO: 6 and SEQ ID NO: 7) were chosen to allow the integration of the H3H and Luz genes in the chloroplast genome. They were amplified by PCR (Polymerase Chain Reaction) from chloroplast DNA of Nicotiana benthamiana. The spectinomycin/streptomycin resistance gene named aadA was amplified by PCR from a plasmid containing it (SEQ ID NO: 19).

To elaborate the plasmid vector containing all these sequences, the plasmid pUC19 was used. The promoters, genes and terminators were amplified by PCR and each part of the trio was ligated together: promoter, gene and terminator with the In-fusion method from Takara. 50 ng or 100 ng of DNA was incubated at 50 °C for 1 h with the ligation enzymes from the In-fusion kit. The ligation products were then amplified by PCR.

The three genes (aadA, H3H, and Luz) thus fused with their respective promoters and terminators, and the trnI and trnA sequences were then cloned into the linearized pUC19 plasmid by PCR, following the NEBuilder protocol.

The NEBuilder is based on the “Gibson assembly” cloning strategy. The primers were designed so that the fragments have a 25 bp sequence overlap with each other and with the sequence of the pUC19 plasmid insertion site.

### Gibson Reaction and Bacterial Transformation

1. 40 to 75 ng of DNA from each PCR product is used with NEBuilder® HiFi DNA assembly 2× buffer and incubated for 1 h at 50 °C. 2. 10-alpha NEB competent bacteria are transformed with the ligation products by heat shock, incubated for 1 h at 37 °C and then plated on LB agar plate with antibiotic and incubated overnight at 37 °C. 3. Plasmid DNA from about 15 clones is extracted and analyzed by sequencing. 4. The positive clones are then amplified in a larger volume of LB+antibiotic (100 ml) and their plasmid DNA is extracted and analyzed by sequencing.

Transformation of Tobacco Plants with the Obtained Plasmids
Coating the Gold Beads with Plasmid DNA

Materials required: 1. 100% ethanol. 2. Sterile gold beads (Biorad). 3. 2.5M CaCl2. 4. 0.1 M spermidine, 5. in vitro plant growth medium: MS with vitamins supplemented with 3% sucrose. 6. Hormones 6-benzyl aminopurine (BAP), indole-3-acetic acid (IAA), indole3-butyric acid (IBA), at the concentration of 1 mg/ml. 7. Spectinomycin at 500 mg/L.

The gold beads are prepared following the Biorad protocol provided with the beads.

The plasmid DNA is then precipitated onto the gold beads (for 5 samples): 1. Vortex 50 μL of gold beads for 1 minute. 2. Add 10 μL of plasmid DNA (at 1 μg/μL) and vortex the mixture. 3. Add 50 μL of 2.5 M Cacl2 and vortex the mixture. 4. Add 20 μL of 0.1 M spermidine and vortex the mixture. The beads are then washed with 100% ethanol and resuspended in 40 ul of 100% ethanol

Bombardment of Nicotiana benthamiana Leaves with Gold Beads

Preparation of the bombardment chamber: 1. wash the chamber and the grids with 70% ethanol 2. Place the gold beads coated with plasmid DNA on the grid provided. 3. Place the intact sheet on Whatman No. 1 filter paper placed on antibiotic-free medium. Place the sample and close the bombardment chamber. 4. Turn on the pump to reach the expected pressure and press the button to fire. 5. Stop the pump to release the pressure and open the chamber. 6. Incubate the bombarded samples on the box for 2 days in the dark. On the third day, cut the explants 3-5 mm square and place them on selection medium (MS supplemented with 3% sucrose and hormones: 1 mg/L BAP, and 0.1 mg/IAA, with 500 mg/L spectinomycin. 3. Transgenic stems appear after 3 to 5 weeks of transformation. Cut the leaves of the emerged transgenic stems into small 2 mm squares and place them in new selection medium, to achieve homoplasmy. Regenerate plants according to known methods.

It can be verified that plant cells produce light when grown on a medium containing hispidin.

### Example 2. Preparation of the Plasmid Containing the H3H, Luz, CPH, HispS, NpgA Genes and the Sequences Allowing the Integration of these Genes into the Chloroplast Genome and their Expression

The sequences of Luz, H3H, CPH, HispS, NpgA genes were adapted with chloroplast usage codons and then synthesized (SEQ ID NO: 1 to SEQ ID NO: 5 respectively). The selected promoters are SEQ ID NO: 9 to SEQ ID NO: 12 respectively and the terminators SEQ ID NO: 14 (Luz and HispS), SEQ ID NO: 15 (H3H), SEQ ID NO: 16 (CPH) and SEQ ID NO: 18 (NpgA).

The trnI and trnA sequences (SEQ ID NO: 6 and SEQ ID NO: 7) were chosen to allow the integration of the H3H and Luz genes into the chloroplast genome. They were amplified by PCR (Polymerase Chain Reaction) from chloroplast DNA of Nicotiana benthamiana. The spectinomycin/streptomycin resistance gene named aadA was amplified by PCR from a plasmid containing it (SEQ ID NO: 19).

An expression cassette was prepared as described above and integrated into a plasmid.

Chloroplast transformation was performed by biolistics on tobacco leaves, as described above.

Samples were recovered, cultured (several times to achieve homoplasmy) on medium containing spectinomycin.

One can thus verify that the cells produce light without the addition of an external compound.

### Example 3 Obtaining Bioluminescent Chloroplasts (from Plant Cells Containing Bioluminescent Chloroplasts

Chloroplasts of Nicotiana benthamiana leaves from shoots were visualized 15 weeks after transformation by bombardment.

For this purpose, several independent transformations were performed on different shoots of Nicotiana benthamiana. On two of them, leaf pieces were taken, which were mounted between slide and coverslip in a drop of sterile miliQ water. As soon as the mounting was obtained, the samples were immediately visualized by microscopy.

Imaging was performed with a Nikon Eclipse Ti microscope with a 100×1.49 NA immersion objective. The 405 nm laser (cw, Oxxius) was used to image the chloroplasts (10 W/cm²). The emission from the samples was spectrally filtered using a dichroic mirror (Di01-R488-25x36, Semrock) and then imaged on a Hamamatsu EM-CCD camera (ImagEM). To detect bioluminescence, the 405 nm laser was blocked with a mechanical shutter. An additional lens was used to obtain a final magnification of 150× corresponding to a pixel size of 106.67 nm. The acquisition time was 1 s.

The results obtained show that chloroplasts exhibit construct-related bioluminescence.

Taking into account the volume of a chloroplast (measurements made with the ImageJ software) which is on average 20 μM² and that a pixel has a surface of 0.01 μM², we can evaluate the global emission of a chloroplast. It is found that the number of photons per second per chloroplast is between 1600 and 3200.

### Example 4 Bioluminescence is Generated by Chloroplast-Encoded Luz and H3H in the Presence of Hispidin in Nicotiana and Petunia Genera

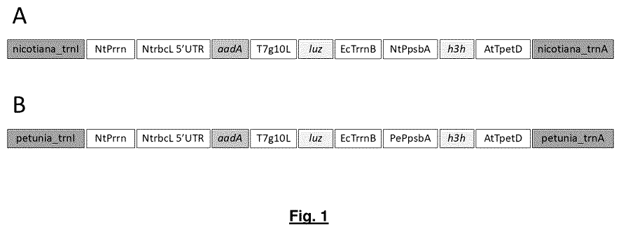

Part A. Preparation of the Plasmid Containing the h3h and Luz Genes and the Sequences Allowing the Integration of these Genes into the Chloroplast Genome, the Transgene Selection and their Expression in Plants Belonging to Nicotiana Genus. luz and h3h genes were inserted into chloroplast genomes of 3 species of Nicotiana genus: Nicotiana tabacum, Nicotiana sylvestris and Nicotiana glauca. The transgene used for chloroplast transformation is presented in FIG. 1A.

As described in the Example 1 of the invention, trnI trnA locus was chosen for the transgene integration. In order to ensure the highest recombination efficiency-dependent on the sequence similarity between the sequences flanking the transgene and the chloroplast genome-trnI and trnA sequences were amplified by PCR from DNA of Nicotiana tabacum, Nicotiana sylvestris, Nicotiana glauca and sequenced. The recovered sequences were identical in the 3 species (SEQ ID NO: 25, SEQ ID NO: 26). trnI locus in Nicotiana benthamiana (SEQ ID NO: 6) differed only by one 9-nt insertion with respect to the homologous regions in other tobacco species (SEQ ID NO: 25) and tmnA locus was identical in all tobacco species. It is to note that shorter sequences were used with respect to Nicotiana benthamiana vector (SEQ ID NO: 25 (970 nt), SEQ ID NO: 26 (873 nt) instead of SEQ ID NO: 6 (1989 nt), SEQ ID NO: 7 (1900 nt)) since they were found to be sufficient to drive homologous recombination.

The expression of aadA (SEQ ID NO: 27) and luz (SEQ ID NO: 28) from Neonothopanus nambi was driven by the same promoter (chloroplastic Prrn promoter, SEQ ID NO: 29). To ensure efficient translation of downstream genes, a chloroplast sequence from rbcL gene was fused to Prrn promoter (SEQ ID NO: 29) and a T7 phage gene 10 leader sequence (T7g10L, SEQ ID NO: 30) was placed between aadA and luz genes. rmnB terminator from Escherichia coli (EcTrrnB SEQ ID NO: 31) was placed downstream of luz gene. The expression of h3h from Neonothopanus nambi (SEQ ID NO: 32) was driven by NtPpsbA promoter and 5′UTR from Nicotiana tabacum (SEQ ID NO: 33) with AtTpetD terminator (3′UTR of petD gene form Arabidopsis thaliana, SEQ ID NO: 34) placed downstream of the gene. The DNA fragments were synthesized or amplified from plant DNA with addition of restriction enzymes that were used to assemble the transgene in pUC19-derived vector. For promoter/gene/terminator fusions type IIS restriction enzymes were used to remove enzyme recognition sites between the DNA fragments.

The vector (FIG. 1A) was used for chloroplast transformation of Nicotiana tabacum, Nicotiana sylvestris and Nicotiana glauca by biolistic delivery using the protocol described in the Example 1 of the invention with the following modifications. After bombardment, the leaf explants were incubated in the dark for 3-4 days on non-selective medium: MS pH 5.8 supplemented with 3% sucrose, vitamins (2 mg/l glycine, 100 mg/l myo-inositol, 0.5 mg/l nicotinic acid, 0.5 mg/l pyridoxine HCl, 0.1 mg/l thiamine HCl), 500 mg/l MES, hormones (1 mg/L BAP, 0.1 mg/l NAA), 2.8 g/l gelrite, then cut into smaller explants and transferred on the same medium supplemented with 500 mg/l spectinomycin. The explants were transferred on fresh selective medium every 2-3 weeks. For Nicotiana glauca, Nicotiana sylvestris and Nicotiana tabacum spectinomycin resistant calli typically appear 3 to 6 months post-bombardment. The resistant calli are propagated every 2-3 weeks on the same medium until shoots develop (typically for 3 to 4 additional months). Spectinomycin resistant shoots are isolated on 0.5× MS pH 5.7 supplemented with 0.5% sucrose, vitamins (1 mg/l glycine, 50 mg/l myo-inositol, 0.25 mg/l nicotinic acid, 0.25 mg/l pyridoxine HCl, 0.05 mg/l thiamine HCl), 250 mg/l MES, 3 g/l gelrite and grown in vitro until roots are developed. The capacity chloroplast-encoded Luz and H3H proteins to generate light was tested by spraying plant material (callus, leaves or plantlets) with hispidin and recording autoluminescence by Amersham Imager 680.

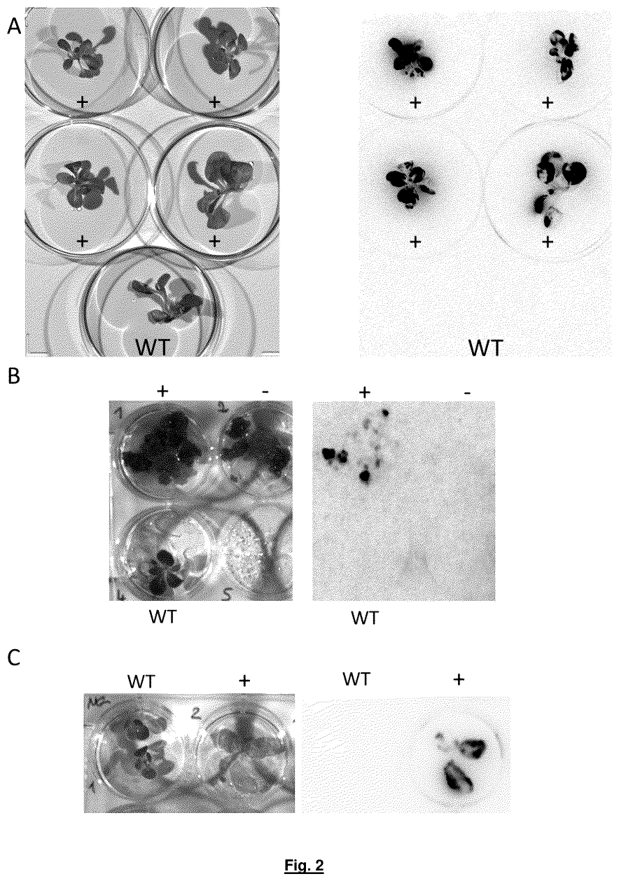

The results presented in FIG. 2 show that Nicotiana tabacum, Nicotiana sylvestris and Nicotiana glauca containing plastome-encoded luz and h3h genes exhibited bioluminescence in the presence of hispidin.

Part B. Preparation of the Plasmid Containing the h3h and Luz Genes and the Sequences Allowing the Integration of these Genes into the Chloroplast Genome, the Transgene Selection in Plants Belonging to Petunia Genus.

A similar vector (FIG. 1B) for luz and h3h insertion in chloroplast genomes of petunia species using aadA (SEQ ID NO: 27) as a selectable marker has been obtained. trnI and trnA loci from Petunia×hybrida Blue Sky, Petunia integrifolia, Petunia exserta and Petunia axillaris were amplified and sequenced. Identical sequences were recovered from the 4 species (petunia_SEQ ID NO: 35 and petunia_trnA SEQ ID NO: 36) and used for the construction of petunia transformation vector. NtPpsbA promoter and 5′UTR driving the expression of h3h gene was replaced with the homologous sequence PePpsbA amplified from Petunia exserta DNA (PePpsbA, SEQ ID NO: 37).

The vector was used for chloroplast transformation of Petunia hybrida, Petunia integrifolia, Petunia exserta and Petunia axillaris by biolistic delivery using the protocol described in the Example 1 of the invention with the following modifications. After bombardment, Petunia leaf explants were incubated in the dark for 3-4 days on non-selective medium: MS pH6 supplemented with 3% sucrose, vitamins (2 mg/l glycine, 100 mg/l myo-inositol, 0.5 mg/l nicotinic acid, 0.5 mg/l pyridoxine HCl, 0.1 mg/l thiamine HCl), 500 mg/l MES, hormones (1 mg/L BAP, 0.1-0.5 mg/l IAA or NAA), 2.8 g/l gelrite, then cut into smaller explants and transferred on the same medium supplemented with 200 mg/l spectinomycin and 200 mg/l streptomycin. In another embodiment, the selection was performed in the presence of 200-500 mg/l spectinomycin and no streptomycin.

The explants are transferred on fresh selective medium every 2-3 weeks until shoots appear. Resistant shoots are isolated on 0.5× MS pH 5.7 supplemented with 0.5% sucrose, vitamins (1 mg/l glycine, 50 mg/l myo-inositol, 0.25 mg/l nicotinic acid, 0.25 mg/l pyridoxine HCl, 0.05 mg/l thiamine HCl), 250 mg/l MES, 3 g/l gelrite. The capacity chloroplast-encoded Luz and H3H proteins to generate light is tested by putting plant material in contact with hispidin and recording autoluminescence by Amersham Imager 680.

The results show that Petunia hybrida, Petunia integrifolia, Petunia exserta and Petunia axillaris exhibit bioluminescence.

1. A plant in which all cells comprising at least one chloroplast are such that the at least one chloroplast comprises at least two constructs each comprising a promoter operably linked to a nucleic acid encoding a protein, wherein the proteins are:
a hispidin-3-hydroxylase (H3H);
a luciferase (Luz),
said plant being selected from the genus Nicotiana or Petunia.

2. The plant according to claim 1, wherein said hispidin-3-hydroxylase (H3H) is a Neonothopanus nambi H3H and/or said luciferase (Luz) is a Neonothopanus nambi Luz.

3. The plant according to claim 1, said plant being selected from Nicotiana tabacum, Nicotiana sylvestris, Nicotiana glauca, Petunia hybrida, Petunia integrifolia, Petunia exserta or Petunia axillaris.

4. The plant according to claim 1, wherein said at least one chloroplast further comprises three constructs each comprising a promoter operably linked to a nucleic acid encoding a protein, wherein the proteins are:
caffeoyl-pyruvate hydrolase (CPH);
hispidin synthase (HispS); and
phosphopantetheinyl transferase (NpgA),
said plant being autoluminescent.

5. The plant according to claim 4, wherein said caffeoyl-pyruvate hydrolase (CPH) is a Neonothopanus nambi CPH and/or said hispidin synthase (HispS) is a Neonothopanus nambi HispS and/or said phosphopantetheinyl transferase (NpgA) is a Aspergillus nidulans NpgA.

6. The plant according to claim 1, wherein the nucleic acids comprise codons adapted for expression in the at least one chloroplast.

7. The plant according to claim 1, wherein the promoters are selected from SEQ ID NO: 8, SEQ ID NO: 9, SEQ ID NO: 10, SEQ ID NO: 11, SEQ ID NO: 12, SEQ ID NO: 29, SEQ ID NO: 33 and SEQ ID NO: 37.

8. The plant according to claim 1, wherein said at least one chloroplast further comprises three constructs each comprising a promoter operably linked to a nucleic acid encoding a protein, wherein the proteins are:
caffeoyl-pyruvate hydrolase (CPH);
hispidin synthase (HispS); and
phosphopantetheinyl transferase (NpgA),
said promoters being selected from SEQ ID NO: 8, SEQ ID NO: 9, SEQ ID NO:
10, SEQ ID NO: 11, SEQ ID NO: 12, SEQ ID NO: 29, SEQ ID NO: 33 and SEQ ID NO: 37,
said plant being autoluminescent.

9. The plant according to claim 1, wherein the constructs are integrated into the genome of the at least one chloroplast at the trnI and trnA sites, represented by SEQ ID NO: 6 or 25 or 35 and SEQ ID NO: 7 or 26 or 36 respectively.

10. The plant according to claim 1, wherein said at least one chloroplast further comprises three constructs each comprising a promoter operably linked to a nucleic acid encoding a protein, wherein the proteins are:
caffeoyl-pyruvate hydrolase (CPH);
hispidin synthase (HispS); and
phosphopantetheinyl transferase (NpgA),
said constructs being integrated into the genome of the at least one chloroplast at the trnI and trnA sites, represented by SEQ ID NO: 6 or 25 or 35 and SEQ ID NO: 7 or 26 or 36 respectively,
said plant being autoluminescent.

11. The plant according to claim 1, wherein the constructs have been integrated into the genome of the at least one chloroplast by homologous recombination.

12. A method of producing the plant according to claim 1, wherein the method comprises:
i. transforming chloroplasts of Nicotiana or Petunia genera plant cell with the at least two constructs and a selection construct comprising a resistance gene selected from the group consisting of: nptII, badh, hph, aadA, cat, aphA6, aacC1, pat, gox, epsp, bxn and als,
wherein the at least two constructs and the selection construct are inserted into the genome of the chloroplasts by homologous recombination,
ii. producing callus from the transformed cell grown on a selective medium comprising:
an antibiotic selected from the group consisting of: neomycin, kanamycin, betain, hygromycin B, spectinomycin, streptomycin, chloramphenicol, amikacin and gentamycin; or
a herbicide selected from the group consisting of: bialophos, phosphinotricin, glufosinate, glyphosate, bromoxynil, sulfonylureas, imidazolines, triazolopyrimidines and pyrimidylbenzoates; and
iii. regenerating a plant from the callus.

13. The method according to claim 10, wherein transformation is performed by bombarding plant leaves using a particle gun.

14. The method according to claim 10, wherein transformation is performed by destabilizing plasma membranes with polyethylene glycol (PEG).

15. The method according to claim 10, wherein the resistance gene is aadA and the selective medium comprises spectinomycin and/or streptomycin.

16. The method according to claim 10, wherein the method further comprises in step i. transforming chloroplasts of Nicotiana or Petunia genera plant cell with additional three constructs each comprising a promoter operably linked to a nucleic acid encoding a protein, wherein the proteins are:
caffeoyl-pyruvate hydrolase (CPH);
hispidin synthase (HispS); and
phosphopantetheinyl transferase (NpgA),
wherein the additional three constructs are inserted into the genome of the chloroplasts by homologous recombination,
said plant being autoluminescent.

17. A method of producing light, wherein the method comprises:
adding hispidin to a culture medium on which the plant is growing; or
spraying hispidin to the plant according to claim 1.


SEQUENCE LISTING (37 sequences)

SEQ ID NO: 1 - DNA, 870 bp, synthetic construct
  misc_feature 1..870: note = optimised Luz sequence
  source 1..870: mol_type = other DNA
  misc_feature 841..867: note = His-tag
atgtcaccacaaacagagactaaagcaagtgttggattcaaaagaattaatatttctttgtcttcactttttgaaagattgagcaagttgtcttcaagatcaattgctataacttgcggagttgttcttgcttctgctatagctttcccaataattagaagagattatcaaactttcttggaagttggaccaagttatgctccccaaaacttcagagggtatattatagtttgtgttctttctttgtttcgacaggaacagaaaggacttgctatatatgatagattgcctgaaaagaggagatggttggctgaccttccatttagggaaggaactagaccatcaattactagtcatattatacaaagacagagaactcaacttgttgatcaagaattcgctaccagagaacttattgataaggttattccaagagttcaagctagacacactgacaaaactttcctttcaacctctaaattcgagtttcatgctaaagctatattccttttgccttcaatacctattaatgaccccttgaatataccatctcatgacactgttagaagaaccaaaagagaaattgcacatatgcacgattatcacgattgcactcttcatttggctcttgctgctcaagatggaaaggaggttttgaaaaaagggtggggacaaagacatccccttgctggaccaggagttccaggaccacctactgaatggactttcctttatgctcccagaaatgaagaagaagctagagttgttgaaatgatagttgaagcttctattgggtatatgactaatgacccagctggaaagattgttgaaaatgctaagggatcttctcatcaccatcatcatcactga

SEQ ID NO: 2 - DNA, 1335 bp, synthetic construct
  misc_feature 1..1335: note = optimised H3h sequence
  source 1..1335: mol_type = other DNA
  misc_feature 1306..1332: note = His-tag
atgtcaccacaaacagagactaaagcaagtgttggattcaaagctagttttgaaaactctctttcagttcttatagttggagctgggttgggagggcttgctgctgctattgctcttagaagacaaggacatgttgttaaaatttatgattcttcaagttttaaggctgaattgggagctggattggctgttcctccaaatactttgagatctttacaacaacttggatgcaacactgaaaatcttaacggagttgataatctttgtttcactgctatgggatatgatgggtctgttggaatgatgaacaatatgactgattatagagaagcttatggaacttcatggattatggttcatagagttgatttgcataatgaattgatgagagttgctcttgatccaggaggattgggacctccagctactttgcatcttaatcatagagttactttctgcgatgttgatgcttgcactgttactttcactaatggaactactcaatcagctgatttgattgttggagctgatgggattagatctactattagaagattcgttttggaagaagatgttactgttccagctagtgggatagttggattcagatggttggttcaagctgatgctcttgatccttatccagaattggattggatagttaagaaaccaccattgggagctagattaattagtactcctcaaaacccacaatctggagttggacttgctgatagaagaactattataatatatgcttgcagaggagggactatggttaacgttttggctgttcatgatgatgaaagagatcaaaacactgctgattggtctgttccagcttcaaaagatgatttgttcagagttttccatgattatcatccaagattcagaagacttttggaattagctcaagatataaacctttggcaaatgagagttgttccagttcttaaaaaatgggttaataaaagagtttgtttacttggagatgctgctcatgcttctcttccaactttgggacaagggttcgggatgggattagaagatgctgttgctttaggaactttgcttccaaaggggactaccgcttcacaaatagaaacccgattggctgtttatgaacaacttagaaaggatcgagctgaattcgttgctgctgaatcatatgaagaacaatatgttccagaaatgcgaggactttatcttagatcaaaggaattgagagatcgagttatgggttatgatataaaagttgaatcagaaaaagttttagaaactttgcttagatcatctaactcagctggatcttctcatcaccatcatcatcactga

SEQ ID NO: 3 - DNA, 936 bp, synthetic construct
  misc_feature 1..936: note = optimised cph sequence
  misc_feature 907..933: note = His-tag
  source 1..936: mol_type = other DNA
atgagaatcaatcctactacttctggttctggggtttccacggctcctattagttcaacttggtctagattaataagattcgttgctattgaaacttcattagttcatataggagaaccaatagatgctactatggatgttggattagctagaagagaaggaaaaactattcaagcttatgaaataatagggtctggatcagctttggatctttctgctcaagtttctaagaacgttcttactgttagagaattgttaatgcctttgtcaagagaagaaataaaaactgttagatgcttgggattgaattatccagttcatgctactgaagctaatgttgctgttcctaagtttccaaatcttttctataaaccagttacttcattgataggaccagatgggttaattactataccttctgttgttcaaccacctaaagaacatcaaagtgattatgaagctgaacttgttatagttataggtaaggctgctaagaatgtttctgaagatgaagctttggattatgttcttggatatactgctgctaatgacatttcatttagaaagcatcaattggctgtttctcaatggagtttttcaaaaggattcgggagtttacttttgactataagaatggctcaaactcattctggaaacattaacagattcagtagagatcaaatattcaatgttaaaaagactatttcatttttatctcaaggtactactttggaaccaggatcaataatattaactggaactccagatggggttggatttgttagaaacccacctctttatttgaaggatggtgatgaagttatgacttggattggatctggaattgggactttagctaatactgttcaagaagaaaaaacttgttttgctagtgggggacatgaaggatcttctcatcaccatcatcatcactga

SEQ ID NO: 4 - DNA, 5103 bp, synthetic construct
  misc_feature 1..5103: note = optimised HispS sequence
  misc_feature 5074..5100: note = His-tag
  source 1..5103: mol_type = other DNA
atgtcaccacaaacagagactaaagcaagtgttggattcaaaaattcttcaaagaatcctccatcaactttgttggatgttttcttggatactgctagaaaccttgatactgctttgagaaacgttcttgaatgcggagaacatagatggagttatagagaattggatactgtttcaagtgctcttgctcaacatcttagatatactgttggattgtctcctactgttgctgttatatctgaaaaccatccatatatattagctcttatgctggctgtttggaagttaggagggacttttgctcctattgatgttcattctcctgctgaattagttgctgggatgttgaatattgttagtccaagttgtcttgttataccttctagtgatgttactaaccaaactcttgcttgcgatcttaacatacctgttgttgctttccatcctcatcaatctactataccagaattgaataagaaatatcttactgattcacaaatatctcctgatttgccattttctgatcctaatagacctgctttgtatcttttcacttcttcagctacttctagaagtaacttaaaatgcgttcctttgactcatacttttatacttagaaactctctttcaaagagagcttggtgtaaaagaatgagaccagaaaccgattttgatgggattagagttttgggatgggctccttggtctcatgttcttgctcatatgcaagatattggacctttgactcttttgaatgctgggtgctatgttttcgctactactccttcaacttatcctactgaattgaaagatgatagagatttgatatcttgtgctgctaacgctattatgtataagggggttaaaagttttgcttgcttaccattcgttcttggggggttgaaggctctttgcgaatctgaaccaagtgttaaggctcatttgcaagttgaagaaagagctcaattgttgaagagcctccagcacatggaaatcttggaatgcggaggagctatgttagaagcttctgttgcttcttgggctattgaaaattgcattcctatatctataggaattgggatgactgaaactggaggggctttatttgctgggcctgtacaagctataaagactggattctcttcagaagataagtttattgaagatgctacttatttacttgtaaaagatgatcatgaatctcatgctgaagaagatataaatgaaggagaacttgttgtaaaaagtaagatgttgcctagaggatatttggggtatagtgatccatctttttcagtagatgatgctgggtgggtaacttttagaactggggatagatattctgtaactccagatggaaagtttagttggttggggagaaacactgattttatacagatgacttcaggagaaactttagatcctagaccaattgaaagttcattgtgtgaatcatcattgatatcaagagcttgcgtaataggggataagtttcttaatggacctgctgctgctgtttgtgctattatagaattagaaccaactgctgttgaaaaaggacaagctcatagtagagaaatagctagagtttttgctccaattaatagagatcttccaccacctttgagaatagcttggagtcatgtattagtacttcaacctagtgaaaaaatacctatgactaaaaaggggaccattttcagaaagaaaattgaacaagttttcgggtctgctttgggaggatcttcaggagataattcacaagcaactgctgatgctggggttgtaagaagagatgaattgtcaaacactgttaaacatattatatcaagagttcttggagtatctgatgatgaattgctttggacccttagtttcgctgaattaggaatgactagtgctcttgctactagaattgctaatgaacttaatgaagtattagttggtgttaacttaccaataaatgcttgctatattcatgttgatcttcctagtttatcaaacgctgtatatgctaagttggctcatttaaaacttccagatagaactccagaaccaagacaagctccagttgaaaactcaggaggtaaagaaatagttgttgttgggcaagctttcagacttcctgggtcaattaacgatgttgcatcattaagagatgctttcttagctagacaagcaagttcaataattactgaaataccatcagatagatgggatcatgcttcattctaccctaaagatataagatttaataaagctggtttagttgatatagctaattatgatcattcattctttgggttgaccgcaactgaagctttgtatctttcacctaccatgagacttgctttggaagtatcatttgaagctttggaaaacgctaacataccagtttcacaattaaagggtagtcaaactgctgtatatgttgcaaccactgacgatgggtttgagacgctcttaaacgctgaagctggttatgatgcatatactagattctatggaactgggagagcagcttctaccgcttctggaagaattagttgtttattggatgtacatggaccatcaattactgttgatactgcttgctctggaggggctgtatgtattgatcaagctatagattatttgcaaagttcatctgctgctgatactgcaattatatgtgcttcaaatactcattgctggccagggtcattcagattcctttctgctcaaggaatggtttcaccaggaggaagatgtgctacttttactaccgatgctgatgggtatgttccaagtgagggtgctgttgctttcattttaaaaactagagaagctgctatgagagataaggatactatattggctactattaaagctactcaaattagtcataacgggagaagtcaaggtttggttgctccaaacgtaaatagtcaagctgatttacatagatcacttttgcaaaaggctggtttgagtccagctgatataagattcattgaagctcatgggaccggaaccagtcttggggatttatcagaaattcaagctattaacgatgcttatacttcttcacaacctagaaccactggaccattaattgtttcagctagtaaaactgttattgggcatactgaacctgctggacctcttgttgggatgctttcagtattgaactcattcaaggaaggggctgtacctgggcttgctcatttgactgctgataacttaaatccatcacttgattgctcatctgtacctttgcttataccttatcaacctgttcatttggctgctccaaaacctcatagagctgctgttagaagttatggatttagtgggaccttggggggaattgtacttgaagctcctgatgaagaaagattagaagaagaacttccaaacgataaacctatgttatttgttgtatctgctaaaacccataccgctttgattgaatatttgggaagatatcttgaatttttattacaagctaacccacaagatttttgcgatatatgttatacctcttgcgtagggagagaacattatagatatagatatgcttgtgttgctaacgatatggaagatcttattgggcaattacaaaagagacttggaagtaaggttccacctaagccttcatataagagaggggctttggcttttgctttctcaggacaagggacccaattcagagggatggctactgaattggctaaagcttatagtgggttcagaaagatagtttctgatttggctaaaagagctagtgaacttagtggtcatgctattgatagattcttgcttgcttatgatataggtgcagaaaacgtagcaccagatagtgaagcagatcaaatttgcatattcgtatatcaatgcagtgttcttagatggttgcaaaccatgggaataagaccatcagctgttatagggcattcattgggagaaatatctgcttcagtagctgctggggctttgtcacttgatagtgctttggatttagtaattagtagagctagattgttaagaagttcagcttcagctcctgcaggaatggctgcaatgtcagctagtcaagatgaagttgtagaacttataggaaagttggatcttgataaagctaattctctttcagtatcagtaattaacgggcctcaaaacactgttgtttctgggtcaagtgctgctatagaatcaattgtagctcttgctaagggtagaaaaattaaggctagtgctcttaacattaaccaagctttccactcaccatatgtagatagtgctgtaccaggattaagagcttggtcagaaaaacatatatcatctgctagaccattacaaattccactttattcaactttacttggtgctcaaatttcagaaggagaaatgcttaacccagatcattgggttgatcatgctagaaaacctgtacaattcgctcaagctgctactaccatgaaggaatcatttaccggagtaattatagatataggaccacaagttgtagcttggtcattgttgctttctaatgggttaacctcagttaccgctttggctgctaagagagggagatcacaacaagttgctttcttgtcagctttagctgatctttatcaagattatggggttgtacctgattttgttggattatatgctcaacaagaagatgcttcaagattaaagaaaactgatattcttacctatccttttcaaagaggtgaagaaactctttcatctgggtcatctactccaaccttagaaaacaccgatttagatagtggtaaagagttacttatggggcctaccagagggcttttgagagctgatgatttgagagattctattgttagtagtgttaaggatgttttggaacttaaatcaaacgaagatttggatctttcagaatctcttaatgctttggggatggattcaattatgtttgctcaattaagaaagagaattggagaaggattagggttgaatgtaccaatggtattcttgtctgatgcattttctataggggaaatggttagtaatttggtagaacaagctgaagcttcagaagataacggatcttctcatcaccatcatcatcactga

SEQ ID NO: 5 - DNA, 1101 bp, synthetic construct
  misc_feature 1..1101: note = optimised NpgA sequence
  misc_feature 1072..1098: note = His-tag
  source 1..1101: mol_type = other DNA
atgtcaccacaaacagagactaaagcaagtgttggattcaaagttcaagatacttcatctgctagtacttctccaattttgactagatggtatatagatactagacctttaactgcttctactgctgctttaccacttttggaaactttgcaacctgctgatcaaatatctgttcaaaagtattatcatttgaaagataaacacatgtcattagcttcaaatttgcttaaatatttattcgttcatagaaactgcagaattccatggtcatctatagttatatcaagaactcctgatcctcatagaagaccttgctatattccaccttcagggtctcaagaagattcttttaaggatggatatactgggataaacgttgaattcaacgtttctcatcaagctagtatggttgctatagctggaactgcttttactcctaattctgggggagattctaagttaaaaccagaagttggaatagatattacttgtgttaatgaaagacaaggaagaaatggagaagaaagaagtttggaatcattgagacaatatattgatatatttagtgaagttttttcaactgctgaaatggctaatattagaagattagatggagtttcatctagttctttatcagctgatagattggttgattatggatatagattattttatacttattgggctcttaaggaagcttatataaaaatgactggggaagctttgttagctccttggttgagagaacttgaatttagtaacgttgttgctccagctgctgttgctgaatctggggattcagctggagatttcggagaaccatatactggggttagaactactctttataagaatcttgtggaagatgttagaatagaagttgctgctttagggggagattatttgtttgctactgctgctagagggggaggaattggggcttcatctagacctggagggggaccagatggatcagggataagatcacaagatccatggagaccatttaaaaagcttgatattgaaagagatattcaaccatgcgctactggagtttgtaactgtctttcaggatcttctcatcaccatcatcatcactga

SEQ ID NO: 6 - DNA, 1989 bp, Nicotiana benthamiana
  source 1..1989: mol_type = other DNA
taccaagctggagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaacctcaaaaacccgtcctcagttcggattgcaggctgcaactcgcctgcatgaagccggaatcgctagtaatcgccggtcagccatacggcggtgaattcgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggatcacctccttttcagggagagctaatgcttgttgggtattttggtttgacactgcttcacacccccaaaaaaaaagaagggagctacgtctgagttaaacttggagatggaagtcttctttcctttctcgacggtgaagtaagaccaagctcatgagcttattatcctaggtcggaacaagttgataggacccccttttttacgtccccatgttccccccgtgtggcgacatgggggcgaaaaaaggaaagagagggatggggtttctctcgcttttggcatagcgggcccccagtgggaggctcgcacgacgggctattagctcagtggtagagcgcgcccctgataattgcgtcgttgtgcctgggctgtgagggctctcagccacatggatagttcaatgtgctcatcggcgcctgaccctgagatgtggatcatccaaggcacattagcatggcgtactcctcctgttcgaaccggggtttgaaaccaaactcctcctcaggaggatagatggggcgattcgggtgagatccaatgtagatccaactttcgattcactcgtgggatccgggcggtccgggggggaccaccacggctcctctcttctcgagaatccatacatcccttatcagtgtatggacagctatctctcgagcacaggtttaggttcggcctcaatgggaaaataaaatggagcacctaacaacgcatcttcacagaccaagaactacgagatcgcccctttcattctggggtgacggagggatcgtaccattcgagccgtttttttcttgactcgaaatgggagcaggtttgaaaaaggatcttagagtgtctagggttgggccaggagggtctcttaacgccttcttttttcttctcatcggagttatttcacaaagacttgccagggtaaggaagaaggggggaacaagcacacttggagagcgcagtacaacggagagttgtatgctgcgttcgggaaggatgaatcgctcccgaaaaggaatctattgattctctcccaattggttggaccgtaggtgcgatgatttacttcacgggcgaggtctctggttcaagtccaggatggccca

SEQ ID NO: 7 - DNA, 1900 bp, Nicotiana benthamiana
  source 1..1900: mol_type = other DNA
gcgccagggaaaagaatagaagaagcatctgactacttcatgcatgctccacttggctcggggggatatagctcagttggtagagctccgctcttgcaattgggtcgttgcgattacgggttggatgtctaattgtccaggcggtaatgatagtatcttgtacctgaaccggtggctcactttttctaagtaatggggaagaggaccgaaacgtgccactgaaagactctactgagacaaagatgggctgtcaagaacgtagaggaggtaggatgggcagttggtcagatctagtatggatcgtacatggacggtagttggagtcggcggctctcccagggttccctcatctgagatctctggggaagaggatcaagttggcccttgcgaacagcttgatgcactatctcccttcaaccctttgagcgaaatgcggcaaaagaaaaggaaggaaaatccatggaccgaccccatcatctccaccccgtaggaactacgagatcaccccaaggacgccttcggcatccaggggtcacggaccgaccatagaaccctgttcaataagtggaacgcattagctgtccgctctcaggttgggcagtcagggtcggagaagggcaatgactcattcttagttagaatgggattccaactcagcaccttttgagtgagattttgagaagagttgctctttggagagcacagtacgatgaaagttgtaagctgtgttcgggggggagttattgtctatcgttggcctctatggtagaatcagtcgggggacctgagaggcggtggtttaccctgcggcggatgtcagcggttcgagtccgcttatctccaactcgtgaacttagccgatacaaagctttatgatagcacccaatttttccgattcggcggttcgatctatgatttatcattcatggacgttgataagatccatccatttagcagcaccttaggatggcatagccttaaaagtgaagggcgaggttcaaacgaggaaaggcttacggtggatacctaggcacccagagacgaggaagggcgtagtaatcgacgaaatgcttcggggagttgaaaataagcatagatccggagattcccgaatagggcaacctttcgaactgctgctgaatccatgggcaggcaagagacaacctggcgaactgaaacatcttagtagccagaggaaaagaaagcaaaagcgattcccgtagtagcggcgagcgaaatgggagcagcctaaaccgtgaaaacggggttgtgggagagcaatacaagcgtcgtgctgctaggcgaagcagcccgaatgctgcaccctagatggcgaaagtccagtagccgaaagcatcactagcttatgctctgacccgagtagcatggggcacgtggaatcccgtgtgaatcagcaaggaccaccttgcaaggctaaatactcctgggtgaccgatagcgaagtagtaccgtgagggaagggtgaaaagaacccccatcggggagtgaaatagaacatgaaaccgtaagctcccaagcagtgggaggagccagggctctgaccgcgtgcctgttgaagaatgagccggcgactcataggcagtggcttggttaagggaacccaccggagccgtagcgaaagcgagtcttcatagggcaattgtcactgcttatggacccgaacctgggtgatctatccatgaccaggatgaagcttgggtgaaactaagtggaggtccgaaccgactgatgttgaagaatcagcggatgagttgtggttaggggtgaaatgccactcgaacccagagctagctggttctccccgaaatgcgttgaggcgcagcagttgactggacatctaggggtaaagcactgtttcggtgcgggccgcgagagcggt

SEQ ID NO: 8 - DNA, 234 bp, synthetic construct
  misc_feature 1..234: note = optimised Prrn promoter
  misc_feature 1..219: note = Promoter
  5'UTR 220..234
  source 1..234: mol_type = other DNA
atcagttcgagcctgattatccctaagcccaatgtgagtttttctagttggatttgctcccccgccgtcgttcaatgagaatggataagaggctcgtgggattgacgtgagggggcagggatggctatatttctgggagcgaactccgggcgaatatgaagcgcatggatacaagttatgccttggaatgaaagacaattccgaatccgctttgtctacgaaggagatagaacc

SEQ ID NO: 9 - DNA, 334 bp, synthetic construct
  misc_feature 1..334: note = optimised PpsbA promoter
  misc_feature 1..276: note = promoter
  5'UTR 277..334
  source 1..334: mol_type = other DNA
atccaagaaaagtgagctattaacgcgtcctattttaatactccgaaggaggcagttggcaggcaactgccactgacgtcccgtaagggtaaggggacgtccactggcgtcccgtaaggggaaggggacgtaggtacataaatgtgctaggtaactaacgtttgattttttgtggtataatatatgtaccatgcttttaatagaagcttgaatttataaattaaaatatttttacaatattttacggagaaattaaaactttaaaaaaattaacatgtcgagtagaccttgttgttgtgagaattcttaattcatgagttgtagggagggattt

SEQ ID NO: 10 - DNA, 379 bp, synthetic construct
  misc_feature 1..379: note = optimised PrbcL promoter
  misc_feature 1..289: note = promoter
  5'UTR 290..379
  source 1..379: mol_type = other DNA
aattccgtatattttcacatctaggatttacatatacaacatataccactgtcaagggggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataatattagtattagttggaaattttgtgaaagattcctatgaaaagtttcattaacacggaattcgtgaattaaccgatcgacgtgcaagcggacatttattttaaattcgataatttttgcaaaaacatttcgacatatttatttattttattatt

SEQ ID NO: 11 - DNA, 277 bp, synthetic construct
  misc_feature 1..277: note = optimised Prrn promoter
  misc_feature 1..219: note = promoter
  5'UTR 220..277
  source 1..277: mol_type = other DNA
atcagttcgagcctgattatccctaagcccaatgtgagtttttctagttggatttgctcccccgccgtcgttcaatgagaatggataagaggctcgtgggattgacgtgagggggcagggatggctatatttctgggagcgaactccgggcgaatatgaagcgcatggatacaagttatgccttggaatgaaagacaattccgaatccgctttgtctacgtcgagtagaccttgttgttgtgagaattcttaattcatgagttgtagggagggattt

SEQ ID NO: 12 - DNA, 306 bp, synthetic construct
  misc_feature 1..306: note = optimised PatpI promoter
  misc_feature 1..248: note = promoter
  5'UTR 249..306
  source 1..306: mol_type = other DNA
tctagctatataagaaatccttgattaataataacataataagataaataacttacttcagaaatcccttatggaatcgcttactatttctgaatttcaaaaaagagataaaaatagctggggatattatgtgatttattagtattctaaatcttagttggtattcaaaatatccgattcaagtagacaaagagatggttgaatcaaaaaattttgtttaaagttcaattttttcagagggcaaggcagtcgagtagaccttgttgttgtgagaattcttaattcatgagttgtagggagggattt

SEQ ID NO: 13 - DNA, 227 bp, synthetic construct
  misc_feature 1..227: note = optimised TpsbA terminator
  source 1..227: mol_type = other DNA
ctagagatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgttt

SEQ ID NO: 14 - DNA, 169 bp, synthetic construct
  misc_feature 1..169: note = optimised Trps16 terminator
  source 1..169: mol_type = other DNA
tcaaccgaaattcaattaaggaaataaattaaggaaatacaaaaaggggggtagtcatttgtatataactttgtatgacttttctcttctatttttttgtatttcctccctttccttttctatttgtatttttttatcattgcttccattgaattccgtgttctataac

SEQ ID NO: 15 - DNA, 173 bp, synthetic construct
  misc_feature 1..173: note = optimised TrbcL terminator
  source 1..173: mol_type = other DNA
taaaaacagtagacattagcagataaattagcaggaaataaagaaaggataaggagaaagaactcaagtaattatccttcgttctcttaattgaattgcaattaaactcggcccaatcttttactaaaaggattgagccgaatacaacaaagattctattgcatatattttga

SEQ ID NO: 16 - DNA, 218 bp, synthetic construct
  misc_feature 1..218: note = optimised TrrnB terminator
  source 1..218: mol_type = other DNA
atcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggctcaaattaagcagaaggccatcctgacggatggcctttttgcgtttctac

SEQ ID NO: 17 - DNA, 228 bp, synthetic construct
  misc_feature 1..228: note = optimised TpsbA terminator
  source 1..228: mol_type = other DNA
tctagagatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgttt

SEQ ID NO: 18 - DNA, 222 bp, synthetic construct
  misc_feature 1..222: note = terminateur TpsbA optimis
  source 1..222: mol_type = other DNA
gatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgttt

SEQ ID NO: 19 - DNA, 792 bp, synthetic construct
  misc_feature 1..792: note = optimised aad gene
  source 1..792: mol_type = other DNA
atgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaatga

SEQ ID NO: 20 - protein, 289 aa, synthetic construct
  REGION 1..289: note = variant of Luz from Neonothopanus nambi
  REGION 281..289: note = His-tag
MSPQTETKASVGFKRINISLSSLFERLSKLSSRSIAITCGVVLASAIAFPIIRRDYQTFLEVGPSYAPQNFRGYIIVCVLSLFRQEQKGLAIYDRLPEKRRWLADLPFREGTRPSITSHIIQRQRTQLVDQEFATRELIDKVIPRVQARHTDKTFLSTSKFEFHAKAIFLLPSIPINDPLNIPSHDTVRRTKREIAHMHDYHDCTLHLALAAQDGKEVLKKGWGQRHPLAGPGVPGPPTEWTFLYAPRNEEEARVVEMIVEASIGYMTNDPAGKIVENAKGSSHHHHHH

SEQ ID NO: 21 - protein, 444 aa, synthetic construct
  REGION 1..444: note = variant of H3H from Neonothopanus nambi
  REGION 436..444: note = His-tag
MSPQTETKASVGFKASFENSLSVLIVGAGLGGLAAAIALRRQGHVVKIYDSSSFKAELGAGLAVPPNTLRSLQQLGCNTENLNGVDNLCFTAMGYDGSVGMMNNMTDYREAYGTSWIMVHRVDLHNELMRVALDPGGLGPPATLHLNHRVTFCDVDACTVTFTNGTTQSADLIVGADGIRSTIRRFVLEEDVTVPASGIVGFRWLVQADALDPYPELDWIVKKPPLGARLISTPQNPQSGVGLADRRTIIIYACRGGTMVNVLAVHDDERDQNTADWSVPASKDDLFRVFHDYHPRFRRLLELAQDINLWQMRVVPVLKKWVNKRVCLLGDAAHASLPTLGQGFGMGLEDAVALGTLLPKGTTASQIETRLAVYEQLRKDRAEFVAAESYEEQYVPEMRGLYLRSKELRDRVMGYDIKVESEKVLETLLRSSNSAGSSHHHHHH

SEQ ID NO: 22 - protein, 311 aa, synthetic construct
  REGION 1..311: note = variant of CPH from Neonothopanus nambi
  REGION 303..311: note = His-tag
MRINPTTSGSGVSTAPISSTWSRLIRFVAIETSLVHIGEPIDATMDVGLARREGKTIQAYEIIGSGSALDLSAQVSKNVLTVRELLMPLSREEIKTVRCLGLNYPVHATEANVAVPKFPNLFYKPVTSLIGPDGLITIPSVVQPPKEHQSDYEAELVIVIGKAAKNVSEDEALDYVLGYTAANDISFRKHQLAVSQWSFSKGFGSLLLTIRMAQTHSGNINRFSRDQIFNVKKTISFLSQGTTLEPGSIILTGTPDGVGFVRNPPLYLKDGDEVMTWIGSGIGTLANTVQEEKTCFASGGHEGSSHHHHHH

SEQ ID NO: 23 - protein, 1700 aa, synthetic construct
  REGION 1..1700: note = variant of HispS from Neonothopanus nambi
  REGION 1692..1700: note = His-tag
MSPQTETKASVGFKNSSKNPPSTLLDVFLDTARNLDTALRNVLECGEHRWSYRELDTVSSALAQHLRYTVGLSPTVAVISENHPYILALMLAVWKLGGTFAPIDVHSPAELVAGMLNIVSPSCLVIPSSDVTNQTLACDLNIPVVAFHPHQSTIPELNKKYLTDSQISPDLPFSDPNRPALYLFTSSATSRSNLKCVPLTHTFILRNSLSKRAWCKRMRPETDFDGIRVLGWAPWSHVLAHMQDIGPLTLLNAGCYVFATTPSTYPTELKDDRDLISCAANAIMYKGVKSFACLPFVLGGLKALCESEPSVKAHLQVEERAQLLKSLQHMEILECGGAMLEASVASWAIENCIPISIGIGMTETGGALFAGPVQAIKTGFSSEDKFIEDATYLLVKDDHESHAEEDINEGELVVKSKMLPRGYLGYSDPSFSVDDAGWVTFRTGDRYSVTPDGKFSWLGRNTDFIQMTSGETLDPRPIESSLCESSLISRACVIGDKFLNGPAAAVCAIIELEPTAVEKGQAHSREIARVFAPINRDLPPPLRIAWSHVLVLQPSEKIPMTKKGTIFRKKIEQVFGSALGGSSGDNSQATADAGVVRRDELSNTVKHIISRVLGVSDDELLWTLSFAELGMTSALATRIANELNEVLVGVNLPINACYIHVDLPSLSNAVYAKLAHLKLPDRTPEPRQAPVENSGGKEIVVVGQAFRLPGSINDVASLRDAFLARQASSIITEIPSDRWDHASFYPKDIRFNKAGLVDIANYDHSFFGLTATEALYLSPTMRLALEVSFEALENANIPVSQLKGSQTAVYVATTDDGFETLLNAEAGYDAYTRFYGTGRAASTASGRISCLLDVHGPSITVDTACSGGAVCIDQAIDYLQSSSAADTAIICASNTHCWPGSFRFLSAQGMVSPGGRCATFTTDADGYVPSEGAVAFILKTREAAMRDKDTILATIKATQISHNGRSQGLVAPNVNSQADLHRSLLQKAGLSPADIRFIEAHGTGTSLGDLSEIQAINDAYTSSQPRTTGPLIVSASKTVIGHTEPAGPLVGMLSVLNSFKEGAVPGLAHLTADNLNPSLDCSSVPLLIPYQPVHLAAPKPHRAAVRSYGFSGTLGGIVLEAPDEERLEEELPNDKPMLFVVSAKTHTALIEYLGRYLEFLLQANPQDFCDICYTSCVGREHYRYRYACVANDMEDLIGQLQKRLGSKVPPKPSYKRGALAFAFSGQGTQFRGMATELAKAYSGFRKIVSDLAKRASELSGHAIDRFLLAYDIGAENVAPDSEADQICIFVYQCSVLRWLQTMGIRPSAVIGHSLGEISASVAAGALSLDSALDLVISRARLLRSSASAPAGMAAMSASQDEVVELIGKLDLDKANSLSVSVINGPQNTVVSGSSAAIESIVALAKGRKIKASALNINQAFHSPYVDSAVPGLRAWSEKHISSARPLQIPLYSTLLGAQISEGEMLNPDHWVDHARKPVQFAQAATTMKESFTGVIIDIGPQVVAWSLLLSNGLTSVTALAAKRGRSQQVAFLSALADLYQDYGVVPDFVGLYAQQEDASRLKKTDILTYPFQRGEETLSSGSSTPTLENTDLDSGKELLMGPTRGLLRADDLRDSIVSSVKDVLELKSNEDLDLSESLNALGMDSIMFAQLRKRIGEGLGLNVPMVFLSDAFSIGEMVSNLVEQAEASEDNGSSHHHHHH

SEQ ID NO: 24 - protein, 366 aa, synthetic construct
  REGION 1..366: note = variant of NpgA from Neonothopanus nambi
  REGION 358..366: note = His-tag
MSPQTETKASVGFKVQDTSSASTSPILTRWYIDTRPLTASTAALPLLETLQPADQISVQKYYHLKDKHMSLASNLLKYLFVHRNCRIPWSSIVISRTPDPHRRPCYIPPSGSQEDSFKDGYTGINVEFNVSHQASMVAIAGTAFTPNSGGDSKLKPEVGIDITCVNERQGRNGEERSLESLRQYIDIFSEVFSTAEMANIRRLDGVSSSSLSADRLVDYGYRLFYTYWALKEAYIKMTGEALLAPWLRELEFSNVVAPAAVAESGDSAGDFGEPYTGVRTTLYKNLVEDVRIEVAALGGDYLFATAARGGGIGASSRPGGGPDGSGIRSQDPWRPFKKLDIERDIQPCATGVCNCLSGSSHHHHHH

SEQ ID NO: 25 - DNA, 970 bp, Nicotiana sp.
  source 1..970: mol_type = other DNA
tctcgacggtgaagtaagaccaagctcatgagcttattatcctaggtcggaacaagttgataggacccccttttttacgtccccatgttccccccgtgtggcgacatgggggcgaaaaaaggaaagagagggatggggtttctctcgcttttggcatagcgggcccccagtgggaggctcgcacgacgggctattagctcagtggtagagcgcgcccctgataattgcgtcgttgtgcctgggctgtgagggctctcagccacatggatagttcaatgtgctcatcggcgcctgaccctgagatgtggatcatccaaggcacattagcatggcgtactcctcctgttcgaaccggggtttgaaaccaaactcctcctcaggaggatagatggggcgattcgggtgagatccaatgtagatccaactttcgattcactcgtgggatccgggcggtccgggggggaccaccacggctcctctcttctcgagaatccatacatcccttatcagtgtatggacagctatctctcgagcacaggtttagcaatgggaaaataaaatggagcacctaacaacgcatcttcacagaccaagaactacgagatcgcccctttcattctggggtgacggagggatcgtaccattcgagccgtttttttcttgactcgaaatgggagcaggtttgaaaaaggatcttagagtgtctagggttgggccaggagggtctcttaacgccttcttttttcttctcatcggagttatttcacaaagacttgccagggtaaggaagaaggggggaacaagcacacttggagagcgcagtacaacggagagttgtatgctgcgttcgggaaggatgaatcgctcccgaaaaggaatctattgattctctcccaattggttggaccgtaggtgcgatgatttacttcacgggcgaggtctctggttcaagtccaggatggcccagctg

SEQ ID NO: 26 - DNA, 873 bp, Nicotiana sp.
  source 1..873: mol_type = other DNA
gctgcgccagggaaaagaatagaagaagcatctgactacttcatgcatgctccacttggctcggggggatatagctcagttggtagagctccgctcttgcaattgggtcgttgcgattacgggttggatgtctaattgtccaggcggtaatgatagtatcttgtacctgaaccggtggctcactttttctaagtaatggggaagaggaccgaaacgtgccactgaaagactctactgagacaaagatgggctgtcaagaacgtagaggaggtaggatgggcagttggtcagatctagtatggatcgtacatggacggtagttggagtcggcggctctcccagggttccctcatctgagatctctggggaagaggatcaagttggcccttgcgaacagcttgatgcactatctcccttcaaccctttgagcgaaatgcggcaaaagaaaaggaaggaaaatccatggaccgaccccatcatctccaccccgtaggaactacgagatcaccccaaggacgccttcggcatccaggggtcacggaccgaccatagaaccctgttcaataagtggaacgcattagctgtccgctctcaggttgggcagtcagggtcggagaagggcaatgactcattcttagttagaatgggattccaactcagcaccttttgagtgagattttgagaagagttgctctttggagagcacagtacgatgaaagttgtaagctgtgttcgggggggagttattgtctatcgttggcctctatggtagaatcagtcgggggacctgagaggcggtggtttaccctgcggcggatgtcagcggttcgagtccgcttatctccaactcgtgaacttagccgatacaaagct

SEQ ID NO: 27 - DNA, 792 bp, Escherichia coli
  source 1..792: mol_type = other DNA
atggcagaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataa

SEQ ID NO: 28 - DNA, 804 bp, Neonothopanus nambi
  source 1..804: mol_type = other DNA
atgagaattaatatttctttgtcttcactttttgaaagattgagcaagttgtcttcaagatcaattgctataacttgcggagttgttcttgcttctgctatagctttcccaataattagaagagattatcaaactttcttggaagttggaccaagttatgctccccaaaacttcagagggtatattatagtttgtgttctttctttgtttcgacaggaacagaaaggacttgctatatatgatagattgcctgaaaagaggagatggttggctgaccttccatttagggaaggaactagaccatcaattactagtcatattatacaaagacagagaactcaacttgttgatcaagaattcgctaccagagaacttattgataaggttattccaagagttcaagctagacacactgacaaaactttcctttcaacctctaaattcgagtttcatgctaaagctatattccttttgccttcaatacctattaatgaccccttgaatataccatctcatgacactgttagaagaaccaaaagagaaattgcacatatgcacgattatcacgattgcactcttcatttggctcttgctgctcaagatggaaaggaggttttgaaaaaagggtggggacaaagacatccccttgctggaccaggagttccaggaccacctactgaatggactttcctttatgctcccagaaatgaagaagaagctagagttgttgaaatgatagttgaagcttctattgggtatatgactaatgacccagctggaaagattgttgaaaatgctaagtaa

SEQ ID NO: 29 - DNA, 163 bp, synthetic construct
  source 1..163: mol_type = other DNA
caatgtgagtttttgtagttggatttgctcccccgccgtcgttcaatgagaatggataagaggctcgtgggattgacgtgagggggcagggatggctatatttctgggagcgaactccgggcgaatatgaagcgcatcgatacaagtgagttgtagggaggga

SEQ ID NO: 30 - DNA, 63 bp, T7 phage
  source 1..63: mol_type = other DNA
gggagaccacaacggtttcccactagaaataattttgtttaactttaagaaggagatataacc

SEQ ID NO: 31 - DNA, 99 bp, Escherichia coli
  source 1..99: mol_type = other DNA
aggcatcaaataaaacgaaaggctcagtcgaaagactgggcctttcgttttatctgttgtttgtcggtgaacgctctcctgagtaggacaaatccgccc

SEQ ID NO: 32 - DNA, 1269 bp, Neonothopanus nambi
  source 1..1269: mol_type = other DNA
atggctagttttgaaaactctctttcagttcttatagttggagctgggttgggagggcttgctgctgctattgctcttagaagacaaggacatgttgttaaaatttatgattcttcaagttttaaggctgaattgggagctggattggctgttcctccaaatactttgagatctttacaacaacttggatgcaacactgaaaatcttaacggagttgataatctttgtttcactgctatgggatatgatgggtctgttggaatgatgaacaatatgactgattatagagaagcttatggaacttcatggattatggttcatagagttgatttgcataatgaattgatgagagttgctcttgatccaggaggattgggacctccagctactttgcatcttaatcatagagttactttctgcgatgttgatgcttgcactgttactttcactaatggaactactcaatcagctgatttgattgttggagctgatgggattagatctactattagaagattcgttttggaagaagatgttactgttccagctagtgggatagttggattcagatggttggttcaagctgatgctcttgatccttatccagaattggattggatagttaagaaaccaccattgggagctagattaattagtactcctcaaaacccacaatctggagttggacttgctgatagaagaactattataatatatgcttgcagaggagggactatggttaacgttttggctgttcatgatgatgaaagagatcaaaacactgctgattggtctgttccagcttcaaaagatgatttgttcagagttttccatgattatcatccaagattcagaagacttttggaattagctcaagatataaacctttggcaaatgagagttgttccagttcttaaaaaatgggttaataaaagagtttgtttacttggagatgctgctcatgcttctcttccaactttgggacaagggttcgggatgggattagaagatgctgttgctttaggaactttgcttccaaaggggactaccgcttcacaaatagaaacccgattggctgtttatgaacaacttagaaaggatcgagctgaattcgttgctgctgaatcatatgaagaacaatatgttccagaaatgcgaggactttatcttagatcaaaggaattgagagatcgagttatgggttatgatataaaagttgaatcagaaaaagttttagaaactttgcttagatcatctaactcagcttaa

SEQ ID NO: 33 - DNA, 224 bp, Nicotiana tabacum
  source 1..224: mol_type = other DNA
gggcaacccactagcatatcgaaattctaattttctgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttacc

SEQ ID NO: 34 - DNA, 132 bp, Arabidopsis thaliana
  source 1..132: mol_type = other DNA
ttcaatttattcaattgtaaaataaacgacgtgggtatctagggaggtatctagggagtagtcatttccaaatgaattctccctagatacatatctaattctaattaatttaattaattaattaaaatgggt

SEQ ID NO: 35 - DNA, 980 bp, Petunia sp.
  source 1..980: mol_type = other DNA
tctcgacggtgaagtaagaccaagctcatgagcttattatcctaggtcggaacaagttgataggacccccttttttacgtccccatgttccccccgtgtggcgacatgggggcgaaaaaaggaaagagagggatggggtttctctcgcttttggcatagcgggcccccagtgggaggctcgcacgacgggctattagctcagtggtagagcgcgcccctgataattgcgtcgttgtgcctgggctgtgagggctctcagccacatggatagttcaatgtgctcatcggcgcctgaccctgagatgtggatcatccaaggcacattagcatggcgtactcctcctgttcgaaccggggtttgaaaccaaactcctcctcaggaggatagatggggcgattcaggtgagatccaatgtagatccaactttcgattcactcgtgggatccgggcggtccgggggggaccaccacggctcctctcttctcgagaatccatacatcccttatcagtgtatggacagctatctctcgagcacaggtttaggttcggcctcaatgggaaaataaaatggagcacctaacaacgcatcttcacagaccaagaactacgagatcacccctttcattctggggtgacggagggatcgtaccattcgagccgtttttttcttgactcgaaatgggagcaggtttgaaaaaggatcttagagtgtctagggttgggccaggagggtctcttaacgccttcttttttcttctcatcggagttattttcacaaagacttgccagggtaaggaagaaggggggaacaagcacacttggagagcgcagtacaacggagagttgtatgctgcgttcgggaaggatgaatcgctcccgaaaaggaatctattgattctctcccaattggttggaccgtaggtgcgatgatttacttcacgggcgaggtctctggttcaagtccaggatggcccagctg

SEQ ID NO: 36 - DNA, 846 bp, Petunia sp.
  source 1..846: mol_type = other DNA
gctgcgccagggaaaagaatagaagaagcatctgactacttcatgcatgctccacttggctcggggggatatagctcagttggtagagctccgctcttgcaattgggtcgttgcgattacgggttggatgtctaattgtccaggcggtaatgatagtatcttgtacctgaaccggtggctcactttttctaagtaatggggaagaggaccgaaacgtgccactgaaagactctactgagacaaagatgggctgtcaagaacgtagaggaggtaggatgggcagttggtcagatctagtatggatcgtacatggacggtagttggagtcggcggctctcccagggttccctcatctgagatctctggggaagaggatcaagttggcccttgcgaacagcttgatgcactatctcccttcaaccctttgagcgaaatgcggcaaaagaaaaggaaggaaaatccatggaccgaccccatcatctccaccccgtaggaactacgagatcaccccaaggacgccttcggcatccaggggtcacggaccgaccatagaaccctgttcaataagtggaacgcattagctgtccgctctcaggttgggcagtcagggtcggagaagggcaatgactcattcttattttgagtgagattttgagaagagttgctctttggagagcacagtacgatgaaagttgtaagctgtgttcgggggggagttattgtctatcgttggcctctatggtagaatcagtcgggggacctgagaggcggtggtttaccctgcggcggatgtcagcggttcgagtccgcttatctccaactcgtgaacttagccgatacaaagct

SEQ ID NO: 37 - DNA, 224 bp, Petunia exserta
  source 1..224: mol_type = other DNA
gggcaacccactatcatatcgaaattcgaattctctgcagagaagtccgtatttttccaatcaacttcattcaaaatttgaatagatctacatacccattggttgacacgagtatataagtcgtgttatactgttgaataacaagccttcctttttctatttttatttgtagaaaactcgtgtgcttgggagtccctgatgattaaataaaccaagattttacc